(19) United States
(12) Patent Application Publication
(10) Pub. No.: US 2025/0010056 A1
(43) Pub. Date: Jan. 9, 2025

(54) INTRAVASCULAR BLOOD PUMPS, MOTORS, AND FLUID CONTROL

(71) Applicant: Shifamed Holdings, LLC, Campbell, CA (US)

(72) Inventors: Daniel VARGHAI, Santa Cruz, CA (US); Ari RYAN, San Jose, CA (US); Harshveer DHALIWAL, San Jose, CA (US)

(21) Appl. No.: 18/711,528

(22) PCT Filed: Nov. 21, 2022
(86) PCT No.: PCT/US2022/080240

§ 371 Date: May 17, 2024

Related U.S. Application Data
(60) Provisional application No. 63/264,355, filed on Nov. 19, 2021.

(51) Int. Cl. A61M 60/416; A61M 60/13; A61M 60/17; A61M 60/216; A61M 60/90

(52) U.S. Cl. CPC A61M 60/416; A61M 60/13; A61M 60/216; A61M 60/90; A61M 60/17; A61M 2205/04; A61M 2205/106; A61M 2205/3327; A61M 2205/3331; A61M 2210/125

(57) ABSTRACT

Intravascular blood pumps systems and methods of use. The blood pump system includes a catheter portion having a distal blood pump with one or more distal collapsible impellers. The system can include a clean purge fluid pathway to carry clean fluid distally to the blood pump and a purge fluid return pathway to carry return fluid proximally into an external motor and out a proximal end of the motor, and optionally to a waste reservoir.

### PRIORITY CLAIM

This patent application claims priority to U.S. provisional patent application No. 63/264,355, titled “INTRAVASCULAR BLOOD PUMPS, MOTORS, AND FLUID CONTROL” and filed on Nov. 19, 2021, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The disclosure is related generally to intravascular blood pumps, motors that are configured to rotate one or more fluid control members such as one or more impellers, and/or one or more fluid pathways therein (e.g., purge fluid, lubricating fluid).

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available can be percutaneously inserted into the vasculature of a patient through an access sheath, thereby not requiring surgical intervention, or through a vascular access graft. A description of this type of device is a percutaneously inserted ventricular support device.

There is a need to provide additional improvements to the field of ventricular support devices and similar blood pumps for treating compromised cardiac blood flow.

### SUMMARY OF THE DISCLOSURE

The present disclosure relates to fluid movement devices, such as intravascular blood pumps, and their methods of use.

One aspect of the disclosure is a blood pump system, comprising: a catheter having a distal end coupled to a blood pump, the blood pump having a driveshaft rotationally coupled to one or more impellers of the blood pump; and a motor assembly having a distal portion coupled to the catheter, the motor assembly including a motor rotationally coupled to the driveshaft, wherein the distal portion of the motor assembly includes a coupling member comprising: one or more fluid channels configured to direct fluid from the motor assembly toward the blood pump via the catheter; and one or more access channels each configured to direct one or more elongate components from the blood pump toward the motor assembly via the catheter.

In this aspect, the one or more elongate components may include one or more of: one or more electrical wires, one or more optical fibers, and one or more air tubes.

In this aspect, the one or more fluid channels and the one or more access channels may be longitudinal cutouts along an outer surface of the coupling member.

In this aspect, the blood pump system may further comprise one or more tube covers fixedly coupled to an outer surface of the coupling member, wherein the one or more fluid channels and the one or more access channels provide spaces between the one or more tube covers and the coupling member.

In this aspect, the catheter may include a drive shaft tube, a catheter shaft tube, and an outer sheath, wherein the drive shaft tube is positioned radially within the catheter shaft tube, and the catheter shaft tube is positioned radially within the outer sheath.

In this aspect, the one or more fluid channels may be configured to direct fluid between the catheter shaft tube and the outer sheath, and the one or more access channels is configured to direct the one or more elongate components between the drive shaft tube and the catheter shaft tube.

In this aspect, the coupling member may be fixedly coupled to a hypotube that surrounds a driveshaft tube, the driveshaft tube accommodating the driveshaft therein.

In this aspect, the distal portion of the motor assembly may further include a coupler assembly, the coupler assembly including a fluid inlet port configured to direct fluid from an external console of the blood pump system toward the coupling member.

In this aspect, the blood pump system may further comprise a second coupling member proximal to the blood pump, the second coupling member including: one or more one or more second fluid channels configured to direct fluid from the catheter toward the blood pump; and one or more second access channels each configured to direct one or more elongate components from the blood pump toward the catheter.

In this aspect, the second one or more access channels may include a sensor holder configured to support one or more sensors therein.

In this aspect, the one or more sensors may include a pressure sensor.

One aspect of the disclosure is a blood pump system, comprising: a motor assembly including a motor rotationally coupled to a driveshaft; a catheter including proximal portion coupled to the motor assembly, the catheter including a distal portion coupled to a blood pump, the blood pump having one or more impellers rotationally coupled to the driveshaft, wherein the distal portion of the catheter includes a coupling member comprising: one or more fluid channels configured to direct fluid from the motor assembly toward the blood pump via the catheter; and one or more access channels each configured to direct one or more elongate components from the blood pump toward the motor assembly via the catheter.

In this aspect, the one or more elongate components may include one or more of: one or more electrical wires, one or more optical fibers, and one or more air tubes.

In this aspect, the one or more access channels may include a sensor holder configured to support one or more sensors therein.

In this aspect, the one or more sensors may include a pressure sensor.

In this aspect, the one or more fluid channels and the one or more access channels may be longitudinal cutouts along an outer surface of the coupling member.

In this aspect, the blood pump system may further comprise one or more tube covers fixedly coupled to an outer surface of the coupling member, wherein the one or more fluid channels and the one or more access channels provide spaces between the one or more tube covers and the coupling member.

In this aspect, the catheter may include a drive shaft tube, a catheter shaft tube, and an outer sheath, wherein the drive shaft tube is positioned radially within the catheter shaft tube, and the catheter shaft tube is positioned radially within the outer sheath.

In this aspect, the one or more fluid channels may be configured to direct fluid from between the catheter shaft tube and the outer sheath toward the blood pump, and the one or more access channels is configured to direct the one or more elongate components between the drive shaft tube and the catheter shaft tube.

In this aspect, the coupling member may be fixedly coupled to a driveshaft tube that surrounds the driveshaft.

A method of manufacturing a blood pump system is also provided, the blood pump system including a catheter having a distal end and a distal end, the method comprising coupling a first coupling member to the proximal end of the catheter and to a distal portion a motor assembly, wherein the coupling member includes: one or more first fluid channels configured to direct fluid from the motor assembly toward the blood pump via the catheter; and one or more first access channels each configured to direct one or more elongate components from a blood pump at a distal end of the catheter toward the motor assembly via the catheter.

In this aspect, the method may further comprise coupling a second coupling member to the distal end of the catheter and to a proximal portion of the blood pump, wherein the second coupling member includes: one or more second fluid channels configured to direct fluid from the motor assembly toward the blood pump via the catheter; and one or more second access channels each configured to direct one or more elongate components from the blood pump toward the motor assembly via the catheter.

In some embodiments, a blood pump system is provided, comprising a handle portion comprising a motor assembly, an elongate catheter extending from the motor assembly, one or more fluid paths disposed in the elongate catheter, a coupling assembly disposed near a distal portion of the elongate catheter, the coupling assembly including a sensor housing, one or more fluid channels coupled to the one or more fluid paths, and a sensor wire channel connecting the sensor housing to the one or more fluid paths.

In some embodiments, the one or more fluid paths comprises a first annular space between a catheter tube of the elongate catheter and a drive cable tube of the elongate catheter.

In one embodiment, the sensor wire channel is configured to receive a sensor wire.

the sensor wire channel is in fluid communication with the first annular space.

In some aspects, the one or more fluid paths are coupled to the first annular space to allow fluid in the first annular space to pass through the coupling assembly.

These and other aspects are described herein.

### BRIEF DESCRIPTION OF THE DRAWINGS

FIG. 1 is a side view of an exemplary pump portion that includes a conduit, a plurality of impellers, an expandable member

FIG. 2 is a side view of an exemplary pump portion that includes a conduit, a plurality of impellers, and a plurality of expandable members.

FIG. 3 illustrates an exemplary placement of a pump portion, the pump portion including a conduit, a plurality of expandable members, and a plurality of impellers.

FIGS. 4A-D illustrates an exemplary blood pump that includes a guidewire pathway and at least one fluid purge pathway.

FIGS. 5A and 5B illustrates an exemplary blood pump that includes a guidewire pathway and at least two fluid purge pathways that are not in fluid communication.

FIGS. 6A-6F illustrate an exemplary sequence of steps that may be carried out based on an exemplary method of using an exemplary blood pump.

FIG. 7A illustrates at least a portion of an exemplary blood pump system including an intravascular blood pump.

FIG. 7B illustrates an exemplary fluid pathway through at least a portion of the exemplary blood pump system of FIG. 7A.

FIGS. 8A and 8B illustrate an exemplary external motor for an intravascular blood pump: FIG. 8A shows a cross-section view of the motor showing an exemplary fluid pathway therethrough; and FIG. 8B shows an external side view of the motor.

FIG. 9 illustrates a cross-section view of an exemplary external motor for an intravascular blood pump showing an exemplary fluid pathway and possible fluid ingress toward a stator motor component.

FIG. 10 illustrates a cross-section view of an exemplary external motor for an intravascular blood pump showing an exemplary isolation component to prevent ingress of fluid toward a stator motor component.

FIGS. 11A-11C illustrate one embodiment of a blood pump system including a pump portion, a bearing assembly, and a catheter portion.

FIGS. 12A-12H illustrate a distal portion of an exemplary motor assembly including a coupling member for managing fluid delivery.

FIGS. 13A-13C illustrate a proximal portion of an exemplary blood pump including a coupling member for managing fluid delivery.

### DETAILED DESCRIPTION

The present disclosure is related to medical devices, systems, and methods of use and manufacture. Medical devices herein may include a pump portion adapted and configured to be disposed within a physiologic vessel, wherein the pump includes one or more components that act upon fluid. For example, pump portions herein may include one or more impellers that are configured such that when rotated, they facilitate the movement of a fluid such as blood.

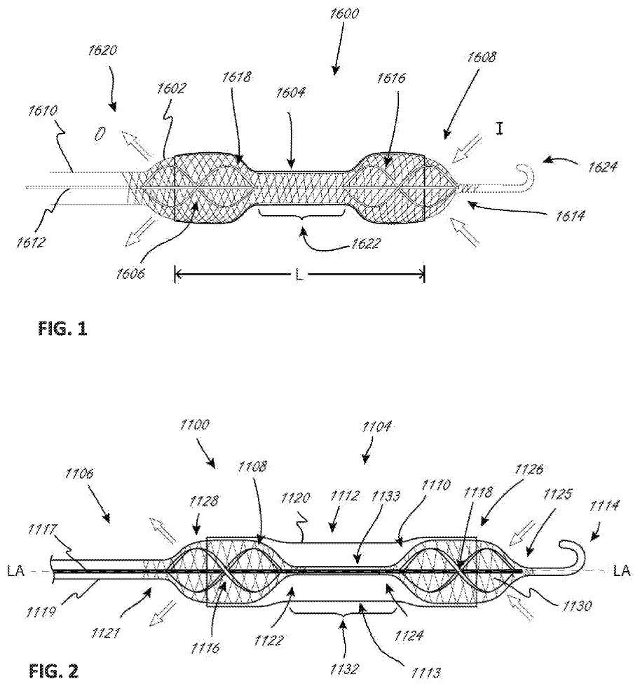

FIG. 1 is a side view illustrating a distal portion of an exemplary intravascular fluid pump, including pump portion 1600, wherein pump portion 1600 includes proximal impeller 1606 and optional distal impeller 1616, both of which are in operable communication with drive cable 1612. Although the embodiment of FIG. 1 shows two impellers (e.g., proximal and distal), it should be understood that other embodiments may include only a single proximal impeller (or alternatively only a single distal impeller). Pump portion 1600 is in an expanded configuration in FIG. 1, but is adapted to be collapsed to a delivery configuration so that it can be delivered with a lower profile. The impellers can in rotational communication with drive cable 1612, directly or indirectly. Drive cable 1612 is in operable communication with an external motor, not shown, and extends through elongate shaft 1610. The phrases “pump portion” and “working portion” (or derivatives thereof) may be used herein interchangeably unless indicated to the contrary. For example without limitation, “pump portion” 1600 can also be referred to herein as a “working portion.”

FIG. 2 is a side view illustrating a deployed configuration (shown extracorporally) of a distal portion of an exemplary embodiment of a fluid movement system. Exemplary system 1100 includes pump portion 1104 (which as set forth herein may also be referred to herein as a pump portion) and an elongate portion 1106 extending from pump portion 1104. Elongate portion 1106 can extend to a more proximal region of the system, not shown for clarity, and that can include, for example, a motor. Pump portion 1104 includes first expandable member 1108 and second expandable member 1110, axially spaced apart along a longitudinal axis LA of pump portion 1104. Spaced axially in this context refers to the entire first expandable member being axially spaced from the entire second expandable member along a longitudinal axis LA of pump portion 1104. A first end 1122 of first expandable member 1108 is axially spaced from a first end 1124 of second expandable member 1110. Some “expandable members” herein may also be referred to herein as baskets.

First and second expandable members 1108 and 1110 generally each include a plurality of elongate segments disposed relative to one another to define a plurality of apertures 1130, only one of which is labeled in the second expandable member 1110. The expandable members can have a wide variety of configurations and can be constructed in a wide variety of ways, such as any of the configurations or constructions in, for example without limitation, U.S. Pat. No. 7,841,976, or the tube in U.S. Pat. No. 6,533,716, which is described as a self-expanding metal endoprosthetic material. For example, without limitation, one or both of the expandable members can have a braided construction or can be at least partially formed by laser cutting a tubular element.

Pump portion 1104 also includes blood flow conduit 1112, which in this embodiment is supported by first expandable member 1108 and to second expandable member 1110. Conduit 1112 also extends axially in between first expandable member 1108 and second expandable member 1110 in the deployed configuration. A central region 1113 of conduit 1112 spans an axial distance 1132 where the pump portion is void of first and second expandable members 1108 and 1110. Central region 1113 can be considered to be axially in between the expandable members. Distal end 1126 of conduit 1112 does not extend as far distally as a distal end 1125 of second expandable member 1110, and proximal end of conduit 1128 does not extend as far proximally as proximal end 1121 of first expandable member 1108.

When the disclosure herein refers to a conduit being coupled to an expandable member, the term coupled in this context does not require that the conduit be directly attached to the expandable member so that conduit physically contacts the expandable member. Even if not directly attached, however, the term coupled in this context refers to the conduit and the expandable member being joined together such that as the expandable member expands or collapses, the conduit also begins to transition to a different configuration and/or size. Coupled in this context therefore refers to conduits that will move when the expandable member to which it is coupled transitions between expanded and collapsed configurations. The conduits herein are considered to create a pathway for fluid to be moved, and may be defined by a one or more components of the pump portion.

Any of the conduits herein can be deformable to some extent. For example, conduit 1112 includes elongate member 1120 that can be made of one or more materials that allow the central region 1113 of conduit to deform to some extent radially inward (towards LA) in response to, for example and when in use, forces from valve tissue (e.g., leaflets) or a replacement valve as pump portion 1104 is deployed towards the configuration shown in FIG. 2. The conduit may be stretched tightly between the expandable members in some embodiments. The conduit may alternatively be designed with a looseness that causes a greater degree of compliance. This can be desirable when the pump portion is disposed across fragile structures such as an aortic valve, which may allow the valve to compress the conduit in a way that minimizes point stresses in the valve. In some embodiments, the conduit may include a membrane attached to the proximal and distal expandable members. Exemplary materials that can be used for any conduits herein include, without limitations, polyurethane rubber, silicone rubber, acrylic rubber, expanded polytetrafluoroethylene, polyethylene, polyethylene terephthalate, including any combination thereof.

Any of the conduits herein can have a thickness of, for example, 0.5-20 thousandths of an inch (thou), such as 1-15 thou, or 1.5 to 15 thou, 1.5 to 10 thou, or 2 to 10 thou.

Any of the conduits herein, or at least a portion of the conduit, can be impermeable to blood. In FIG. 2, pump portion 1104 includes a lumen that extends from distal end 1126 of conduit 1112 and extends to proximal end 1128 of conduit 1112. The lumen is defined by conduit 1112 in central region 1113, but can be thought of being defined by both the conduit and portions of the expandable members in regions axially adjacent to central region 1113. In this embodiment, however, it is the conduit material that causes the lumen to exist and prevents blood from passing through the conduit.

Any of the conduits herein that are secured to one or more expandable members can be, unless indicated to the contrary, secured so that the conduit is disposed radially outside of one or more expandable members, radially inside of one or more expandable members, or both, and the expandable member can be impregnated with the conduit material.

The proximal and distal expandable members help maintain the conduit in an open configuration by providing radial support for the conduit, while each also creates a working environment for an impeller, described below. Each of the expandable members, when in the deployed configuration, is maintained in a spaced relationship relative to a respective impeller, which allows the impeller to rotate within the expandable member without contacting the expandable member. Pump portion 1104 can include first impeller 1116 and optional second impeller 1118, with first impeller 1116 disposed radially within first expandable member 1108 and optional second impeller 1118 disposed radially within second expandable member 1110. In embodiments with two impellers, the two impellers even though they are distinct and separate impellers, are in operable communication with a common drive mechanism (e.g., drive cable 1117), such that when the drive mechanism is activated the two impellers rotate together. In embodiments where the pump includes only a single proximal impeller or single distal impeller, the drive mechanism is in operable communication with the impeller. In this deployed configuration, impellers 1116 and 1118 are axially spaced apart along longitudinal axis LA, just as are the expandable members 1108 and 1110 are axially spaced apart.

Impellers 1116 and 1118 are also axially within the ends of expandable members 1108 and 1110, respectively (in addition to being radially within expandable members 1108 and 1110). The impellers herein can be considered to be axially within an expandable member even if the expandable member includes struts extending from a central region of the expandable member towards a longitudinal axis of the pump portion (e.g., tapering struts in a side view). In FIG. 2, second expandable member 1110 extends from first end 1124 (proximal end) to second end 1125 (distal end).

In FIG. 2, a distal portion of impeller 1118 extends distally beyond distal end 1126 of conduit 1112, and a proximal portion of impeller 1116 extends proximally beyond proximal end 1128 of conduit 1112. In this FIG., portions of each impeller are axially within the conduit in this deployed configuration.

In the exemplary embodiment shown in FIG. 2, impellers 1116 and 1118 are in operable communication with a common drive mechanism 1117, and in this embodiment, the impellers are each coupled to drive mechanism 1117, which extends through shaft 1119 and pump portion 1104. Drive mechanism 1117 can be, for example, an elongate drive cable, which when rotated causes the impellers to rotate. In this example, as shown, drive mechanism 1117 extends to and is axially fixed relative to distal tip 1114, although it is adapted to rotate relative to distal tip 1114 when actuated. Thus, in this embodiment, the impellers and drive mechanism 1117 rotate together when the drive mechanism is rotated. Any number of known mechanisms can be used to rotate drive mechanism, such as with a motor (e.g., an external motor).

The expandable members and the conduit are not in rotational operable communication with the impellers and the drive mechanism. In this embodiment, proximal end 1121 of proximal expandable member 1108 is coupled to shaft 1119, which may be a shaft of elongate portion 1106 (e.g., an outer catheter shaft). Distal end 1122 of proximal expandable member 1108 is coupled to central tubular member 1133, through which drive mechanism 1117 extends. Central tubular member 1133 extends distally from proximal expandable member 1108 within conduit 1112 and is also coupled to proximal end 1124 of distal expandable member 1110. Drive mechanism 1117 thus rotates within and relative to central tubular member 1133. Central tubular member 1133 extends axially from proximal expandable member 1108 to distal expandable member 1110. Distal end 1125 of distal expandable member 1110 is coupled to distal tip 1114, as shown. Drive mechanism 1117 is adapted to rotate relative to tip 1114, but is axially fixed relative to tip 1114.

Pump portion 1104 is adapted and configured to be collapsed to a smaller profile than its deployed configuration (which is shown in FIG. 2). This allows it to be delivered using a lower profile delivery device (smaller French size) than would be required if none of pump portion 1104 was collapsible. Even if not specifically stated herein, any of the expandable members and impellers may be adapted and configured to be collapsible to some extent to a smaller delivery configuration.

The pump portions herein can be collapsed to a collapsed delivery configuration using conventional techniques, such as with an outer sheath that is movable relative to the pump portion (e.g., by axially moving one or both of the sheath and pump portion). For example without limitation, any of the systems, devices, or methods shown in the following references may be used to facilitate the collapse of a pump portion herein: U.S. Pat. No. 7,841,976 or U.S. Pat. No. 8,052,749, the disclosures of which are incorporated by reference herein for all purposes.

In alternative embodiments, at least a portion of any of the impellers herein may extend outside of the fluid lumen. For example, only a portion of an impeller may extend beyond an end of the fluid lumen in either the proximal or distal direction. In some embodiments, a portion of an impeller that extends outside of the fluid lumen is a proximal portion of the impeller, and includes a proximal end (e.g., see the proximal impeller in FIG. 2). In some embodiments, the portion of the impeller that extends outside of the fluid lumen is a distal portion of the impeller, and includes a distal end (e.g., see the distal impeller in FIG. 2). When the disclosure herein refers to impellers that extend outside of the fluid lumen (or beyond an end), it is meant to refer to relative axial positions of the components, which can be most easily seen in side views or top views, such as in FIG. 2.

An optional second impeller at another end of the fluid lumen may not, however, extend beyond the fluid lumen. For example, an illustrative alternative design can include a proximal impeller that extends proximally beyond a proximal end of the fluid lumen (like the proximal impeller in FIG. 2), and the fluid lumen does not extend distally beyond a distal end of a distal impeller. Alternatively, a distal end of a distal impeller can extend distally beyond a distal end of the fluid lumen, but a proximal end of a proximal impeller does not extend proximally beyond a proximal end of the fluid lumen. In any of the pump portions herein, none of the impellers may extend beyond ends of the fluid lumen.

While specific exemplary locations may be shown herein, the fluid pumps may be able to be used in a variety of locations within a body. Some exemplary locations for placement include placement in the vicinity of an aortic valve or pulmonary valve, such as spanning the valve and positioned on one or both sides of the valve, and in the case of an aortic valve, optionally including a portion positioned in the ascending aorta. In some other embodiments, for example, the pumps may be, in use, positioned further downstream, such as being disposed in a descending aorta.

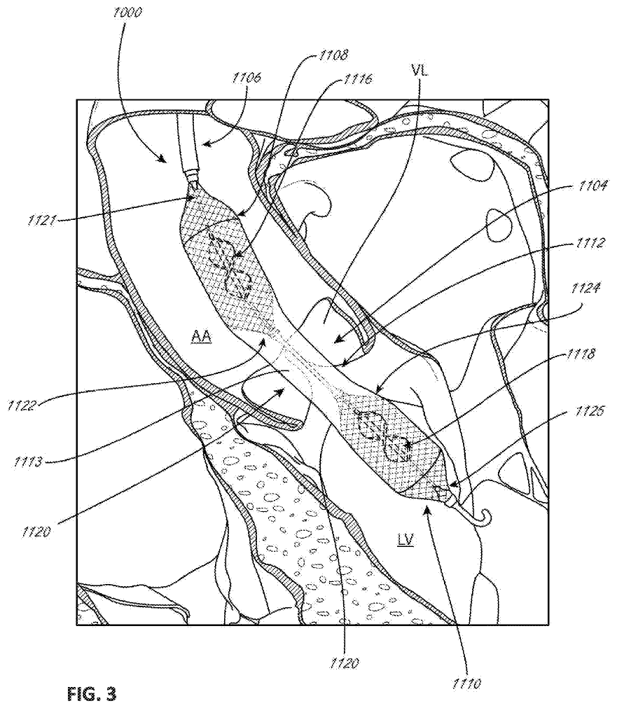

FIG. 3 illustrates an exemplary placement of pump portion 1104 from system 1000 from FIG. 2, and also illustrates an exemplary placement location for any of the pump portions herein. One difference shown in FIG. 3 is that the conduit extends at least as far as the ends of the impellers. FIG. 3 shows pump portion 1104 in a deployed configuration, positioned in place across an aortic valve. Pump portion 1104 can be delivered as shown via, for example without limitation, femoral artery access (a known access procedure). While not shown for clarity, system 1000 can also include an outer sheath or shaft in which pump portion 1104 is disposed during delivery to a location near an aortic valve. The sheath or shaft can be moved proximally (towards the ascending aorta “AA” and away from left ventricle “LV”) to allow for deployment and expansion of pump portion 1104. For example, the sheath can be withdrawn to allow for expansion of second expandable member 1110, with continued proximal movement allowing first expandable member 1108 to expand.

In this embodiment, second expandable member 1110 has been expanded and positioned in a deployed configuration such that distal end 1125 is in the left ventricle “LV,” and distal to aortic valve leaflets “VL,” as well as distal to the annulus. Proximal end 1124 has also been positioned distal to leaflets VL, but in some methods proximal end 1124 may extend slightly axially within the leaflets VL. This embodiment is an example of a method in which at least half of the second expandable member 1110 is within the left ventricle, as measured along its length (measured along the longitudinal axis). And as shown, this is also an example of a method in which the entire second expandable member 1110 is within the left ventricle. This is also an example of a method in which at least half of second impeller 1118 is positioned within the left ventricle, and also an embodiment in which the entire second impeller 1118 is positioned within the left ventricle. It should be understood, however, that some embodiments include only a single impeller (e.g., only proximal impeller 1116) and no second impeller 1118 positioned within the left ventricle.

Continued retraction of an outer shaft or sheath (and/or distal movement of working end 1104 relative to an outer sheath or shaft) continues to release conduit 1112, until central region 1113 is released and deployed. The expansion of expandable members 1108 and 1110 causes conduit 1112 to assume a more open configuration, as shown in FIG. 3. Thus, while in this embodiment conduit 1112 does not have the same self-expanding properties as the expandable members, the conduit will assume a deployed, more open configuration when the working end is deployed. At least a portion of central region 1113 of conduit 1112 is positioned at an aortic valve coaptation region.

Continued retraction of an outer shaft or sheath (and/or distal movement of working end 1104 relative to an outer sheath or shaft) deploys first expandable member 1108. In this embodiment, first expandable member 1108 has been expanded and positioned (as shown) in a deployed configuration such that proximal end 1121 is in the ascending aorta AA, and proximal to leaflets “VL.” Distal end 1122 has also been positioned proximal to leaflets VL, but in some methods distal end 1122 may extend slightly axially within the leaflets VL. This embodiment is an example of a method in which at least half of first expandable member 1110 is within the ascending aorta, as measured along its length (measured along the longitudinal axis). And as shown, this is also an example of a method in which the entire first expandable member 1110 is within the AA. This is also an example of a method in which at least half of first impeller 1116 is positioned within the AA, and also an embodiment in which the entire first impeller 1116 is positioned within the AA.

At any time during or after deployment of pump portion 1104, the position of the pump portion can be assessed in any way, such as under fluoroscopy. The position of the pump portion can be adjusted at any time during or after deployment. For example, after second expandable member 1110 is released but before first expandable member 1108 is released, pump portion 1104 can be moved axially (distally or proximally) to reposition the pump portion. Additionally, for example, the pump portion can be repositioned after the entire working portion has been released from a sheath to a desired final position.

It is understood that the positions of the components (relative to the anatomy) shown in FIG. 3 are considered exemplary final positions for the different components of working portion 1104, even if there was repositioning that occurred after initial deployment.

The one or more expandable members herein can be configured to be, and can be expanded in a variety of ways, such as via self-expansion, mechanical actuation (e.g., one or more axially directed forces on the expandable member, expanded with a separate balloon positioned radially within the expandable member and inflated to push radially outward on the expandable member), or a combination thereof.

Expansion as used herein refers generally to reconfiguration to a larger profile with a larger radially outermost dimension (relative to the longitudinal axis), regardless of the specific manner in which the one or more components are expanded. For example, a stent that self-expands and/or is subject to a radially outward force can “expand” as that term is used herein. A device that unfurls or unrolls can also assume a larger profile, and can be considered to expand as that term is used herein.

The one or more impellers can similarly be adapted and configured to be, and can be expanded in a variety of ways depending on their construction. For examples, one or more impellers can, upon release from a sheath, automatically revert to or towards a different larger profile configuration due to the material(s) and/or construction of the impeller design (see, for example, U.S. Pat. No. 6,533,716, or U.S. Pat. No. 7,393,181, both of which are incorporated by reference herein for all purposes). Retraction of an outer restraint can thus, in some embodiments, allow both the expandable member and the impeller to revert naturally to a larger profile, deployed configuration without any further actuation.

As shown in the example in FIG. 3, the pump portion includes a first (proximal) impeller and an option second (distal) impeller that may be spaced on either side of an aortic valve, each disposed within a separate expandable member. This is in contrast to some designs in which a working portion includes a single elongate expandable member (such as in FIG. 1). Rather than a single generally tubular expandable member extending all the way across the valve, the illustrated example has a working end 1104 includes a conduit 1112 extending between expandable members 1108 and 1110. The conduit is more flexible and deformable than the pump at the locations of the impellers, which can allow for more deformation of the pump portion at the location of the leaflets than would occur if an expandable member spanned the aortic valve leaflets. Having a more flexible central region may also cause less damage to the leaflets after the pump portion has been deployed in the subject.

Additionally, forces on a central region of a single expandable member from the leaflets might translate axially to other regions of the expandable member, perhaps causing undesired deformation of the expandable member at the locations of the one or more impellers. This may cause the outer expandable member to contact the impeller, undesirably interfering with the rotation of the impeller. Designs that include separate expandable members around each impeller, particularly where each expandable member and each impeller are supported at both ends (i.e., distal and proximal), result in a high level of precision in locating the impeller relative to the expandable member. Two separate expandable members may be able to more reliably retain their deployed configurations compared with a single expandable member.

Embodiments herein can thus achieve a smaller delivery profile while maintaining sufficiently high flow rates, while creating a more deformable and flexible central region of the working portion, the exemplary benefits of which are described above (e.g., interfacing with delicate valve leaflets).

Any of the conduits herein act to, are configured to, and are made of material(s) that create a fluid lumen therein between a first end (e.g., distal end) and a second end (e.g., proximal end). Fluid flows into the inflow region, through the fluid lumen, and then out of an outflow region. Flow into the inflow region may be labeled herein as “I,” and flow out at the outflow region may be labeled “O.” Any of the conduits herein can be impermeable. Any of the conduits herein can alternatively be semipermeable. Any of the conduits herein may also be porous, but will still define a fluid lumen therethrough. In some embodiments the conduit is a membrane, or other relatively thin layered member. Any of the conduits herein, unless indicated to the contrary, can be secured to an expandable member such that the conduit, where is it secured, can be radially inside and/or outside of the expandable member. For example, a conduit can extend radially within the expandable member so that inner surface of the conduit is radially within the expandable member where it is secured to the expandable member.

Any of the expandable member(s) herein can be constructed of a variety of materials and in a variety of ways. For example, the expandable member may have a braided construction, or it can be formed by laser machining. The material can be deformable, such as nitinol. The expandable member can be self-expanding or can be adapted to be at least partially actively expanded.

In some embodiments, the expandable member is adapted to self-expand when released from within a containing tubular member such as a delivery catheter, a guide catheter or an access sheath. In some alternative embodiments, the expandable member is adapted to expand by active expansion, such as action of a pull-rod that moves at least one of the distal ends and the proximal end of the expandable member toward each other. In alternative embodiments, the deployed configuration can be influenced by the configuration of one or more expandable structures. In some embodiments, the one or more expandable members can be deployed, at least in part, through the influence of blood flowing through the conduit. Any combination of the above mechanisms of expansion may be used.

The blood pumps and fluid movement devices, system and methods herein can be used and positioned in a variety of locations within a body. While specific examples may be provided herein, it is understood that that the working portions can be positioned in different regions of a body than those specifically described herein.

In any of the embodiments or in any part of the description herein that include a distal impeller and a proximal impeller, the axial spacing between a distal end of the proximal impeller and a proximal end of the distal impeller can be from 1.5 cm to 25 cm (inclusive) along a longitudinal axis of the pump portion, or along a longitudinal axis of a housing portion that includes a fluid lumen. The distance may be measured when the pump portion, including any impellers, is in an expanded configuration. This exemplary range can provide the exemplary flexibility benefits described herein as the pump portion is delivered through curved portions of the anatomy, such as, for example, an aortic valve via an aorta. In embodiments in which there may be more than two impellers, any two adjacent impellers (i.e., impellers that do not have any other rotating impeller in between them) may be spaced axially by any of the axial spacing distances described herein.

While some embodiments include a proximal impeller distal end that is axially spaced 1.5 cm to 25 cm from a distal impeller proximal end along an axis, the disclosure herein also includes any axial spacings that are subranges within that general range of 1.5 cm to 25 cm. That is, the disclosure includes all ranges that have any lower limit from 1.5 and above in that range, and all subranges that have any upper limit from 25 cm and below. The examples below provide exemplary subranges. In some embodiments, a proximal impeller distal end is axially spaced 1.5 cm to 20 cm from a distal impeller proximal end along an axis, 1.5 cm to 15 cm, 1.5 cm to 10 cm, 1.5 cm to 7.5 cm, 1.5 cm to 6 cm, 1.5 cm to 4.5 cm, 1.5 cm to 3 cm. In some embodiments the axial spacing is 2 cm to 20 cm, 2 cm to 15 cm, 2 cm to 12 cm, 2 cm to 10 cm, 2 cm to 7.5 cm, 2 cm to 6 cm, 2 cm to 4.5 cm, 2 cm to 3 cm. In some embodiments the axial spacing is 2.5 cm to 15 cm, 2.5 cm to 12.5 cm, 2.5 cm to 10 cm, 2.5 cm to 7.5 cm, or 2.5 cm to 5 cm (e.g., 3 cm). In some embodiments the axial spacing is 3 cm to 20 cm, 3 cm to 15 cm, 3 cm to 10 cm, 3 cm to 7.5 cm, 3 cm to 6 cm, or 3 cm to 4.5 cm. In some embodiments the axial spacing is 4 cm to 20 cm, 4 cm to 15 cm, 4 cm to 10 cm, 4 cm to 7.5 cm, 4 cm to 6 cm, or 4 cm to 4.5 cm. In some embodiments the axial spacing is 5 cm to 20 cm, 5 cm to 15 cm, 5 cm to 10 cm, 5 cm to 7.5 cm, or 5 cm to 6 cm. In some embodiments the axial spacing is 6 cm to 20 cm, 6 cm to 15 cm, 6 cm to 10 cm, or 6 cm to 7.5 cm. In some embodiments the axial spacing is 7 cm to 20 cm, 7 cm to 15 cm, or 7 cm to 10 cm. In some embodiments the axial spacing is 8 cm to 20 cm, 8 cm to 15 cm, or 8 cm to 10 cm. In some embodiments the axial spacing is 9 cm to 20 cm, 9 cm to 15 cm, or 9 cm to 10 cm. In various embodiments, the fluid lumen between the impellers is relatively unsupported.

In any of the embodiments herein the one or more impellers may have a length, as measured axially between an impeller distal end and an impeller proximal end, from 0.5 cm to 10 cm, or any subrange thereof. The examples below provide exemplary subranges. In some embodiments the impeller axial length is from 0.5 cm to 7.5 cm, from 0.5 cm to 5 cm, from 0.5 cm to 4 cm, from 0.5 cm to 3 cm, from 0.5 cm to 2, or from 0.5 cm to 1.5 cm. In some embodiments the impeller axial length is from 0.8 cm to 7.5 cm, from 0.8 cm to 5 cm, from 0.8 cm to 4 cm, from 0.8 cm to 3 cm, from 0.8 cm to 2 cm, or from 0.8 cm to 1.5 cm. In some embodiments the impeller axial length is from 1 cm to 7.5 cm, from 1 cm to 5 cm, from 1 cm to 4 cm, from 1 cm to 3 cm, from 1 cm to 2 cm, or from 1 cm to 1.5 cm. In some embodiments the impeller axial length is from 1.2 cm to 7.5 cm, from 1.2 cm to 5 cm, from 1.2 cm to 4 cm, from 1.2 cm to 3 cm, from 1.2 to 2 cm, or from 1.2 cm to 1.5 cm. In some embodiments the impeller axial length is from 1.5 cm to 7.5 cm, from 1.5 cm to 5 cm, from 1.5 cm to 4 cm, from 1.5 cm to 3 cm, or from 1.5 cm to 2 cm. In some embodiments the impeller axial length is from 2 cm to 7.5 cm, from 2 cm to 5 cm, from 2 cm to 4 cm, or from 2 cm to 3 cm. In some embodiments the impeller axial length is from 3 cm to 7.5 cm, from 3 cm to 5 cm, or from 3 cm to 4 cm. In some embodiments the impeller axial length is from 4 cm to 7.5 cm, or from 4 cm to 5 cm.

In any of the embodiments herein the fluid lumen can have a length from a distal end to a proximal end. In some embodiments the fluid lumen length is from 4 cm to 40 cm, or any subrange therein. For example, in some embodiments the length can be from 4 cm to 30 cm, from 4 cm to 20 cm, from 4 cm to 18 cm, from 4 cm to 16 cm, from 4 cm to 14 cm, from 4 cm to 12 cm, from 4 cm to 10 cm, from 4 cm to 8 cm, from 4 cm to 6 cm.

In any of the embodiments herein the housing can have a deployed diameter, at least the location of an impeller (and optionally at a location between impellers. In some embodiments the deployed diameter can be from 0.3 cm to 1.5 cm, or any subrange therein. For example, Dp may be from 0.4 cm to 1.4 cm, from 0.4 cm to 1.2 cm, from 0.4 cm to 1.0 cm, from 0.4 cm to 0.8 cm, or from 0.4 cm to 0.6 cm. In some embodiments, the deployed diameter may be from 0.5 cm to 1.4 cm, from 0.5 cm to 1.2 cm, from 0.5 cm to 1.0 cm, from 0.5 cm to 0.8 cm, or from 0.5 cm to 0.6 cm. In some embodiments the deployed diameter may be from 0.6 cm to 1.4 cm, from 0.6 cm to 1.2 cm, from 0.6 cm to 1.0 cm, or from 0.6 cm to 0.8 cm. In some embodiments the deployed diameter may be from 7 cm to 1.4 cm, from 0.7 cm to 1.2 cm, from 0.7 cm to 1.0 cm, or from 0.7 cm to 0.8 cm.

In any of the embodiments herein an impeller can have a deployed diameter. In some embodiments the impeller deployed diameter can be from 1 mm-30 mm, or any subrange therein. For example, in some embodiments the impeller deployed diameter may be from 1 mm-15 mm, from 2 mm-12 mm, from 2.5 mm-10 mm, or 3 mm-8 mm.

In any of the embodiments herein, a tip gap exists between an impeller outer diameter and a fluid lumen inner diameter. In some embodiments the tip gap can be from 0.01 mm-1 mm, such as 0.05 mm to 0.8 mm, or such as 0.1 mm-0.5 mm.

In any of the embodiments herein, at least one of a flow diffuser or diffusers and a stator or stators is/are located between two or more impellers along the catheter shaft, any one of which can increase fluid pressure between impellers, reduce swirl of the fluid, and/or increase the efficiency of the multiple impellers as a group.

In any of the embodiments herein, features at the fluid exit of an expandable shroud basket or expandable member are shaped to act as a flow diffuser, such as stent-like struts at the attachments between the catheter shaft outer dimension and the expandable member outer dimension, which can be blade-shaped with a twist directed to change the flow direction of blood. In any of the embodiments herein, one or more portions of the catheter shaft downstream of an impeller may flare to a larger diameter to change the angle of blood flow and cause deceleration of the blood flow to a speed closer to native aortic blood flow. Exemplary locations for a larger diameter downstream of an impeller would be at or near the area where an expandable shroud basket attaches to the catheter shaft, and/or at a bearing housing adjacent the impeller, or on or adjacent an internal motor.

In some embodiments, the pump portion can include one or more central members disposed axially in between proximal and distal impellers. The one or more central members may be coupled directly to one another, or they may not. The one or more central members may provide one or more of the following exemplary functions: structural support, flow modification, and maintaining impeller alignment. If the one or more central members provide structural support, the one or more central members may provide structural support to the outer conduit (which may be referred to herein as a “housing”) and/or to one or more impellers. For example, they may help maintain tip gap in one or more impellers. In the description that follows, the one or more central members are not in rotational operation with an impeller, unless indicated to the contrary. As used herein, the term “central member” or derivatives thereof does not imply that the member is located at least a midpoint between two impellers, but simply that the central member is somewhere axially between the two impellers. “Central member” may thus be used interchangeably herein with the term “intermediate member.”

While some of the embodiments above describe pump portions or components that are collapsible and expandable (or at least movable between collapsed and expanded configurations), in any of those embodiments the components and expandable outer housing may also be non-expandable and non-collapsible. That is, any of the components in those embodiments may be present, but the components may be non-expandable variations of those components. For example, the impellers above may be non-expandable rather than expandable.

Blood pumps, such as any of the intravascular pumps herein, may benefit from having one or more fluid paths through which fluid can flow through the device. For example without limitation, blood pumps may benefit from having one or more fluid paths through which fluid can flow to perform any of these exemplary functions: cooling rotating components (e.g., a drive cable) to prevent their overheating; flushing small particulates that may break off rotating components (e.g., a drive cable) to prevent the rotating parts from being damaged by the small particulates; lubricating rotating components (e.g., one or more bearings), and preventing blood ingress into the pump (e.g., near or at a distal end of the pump). Fluid delivery through the one or more flow paths may provide any number of these functions.

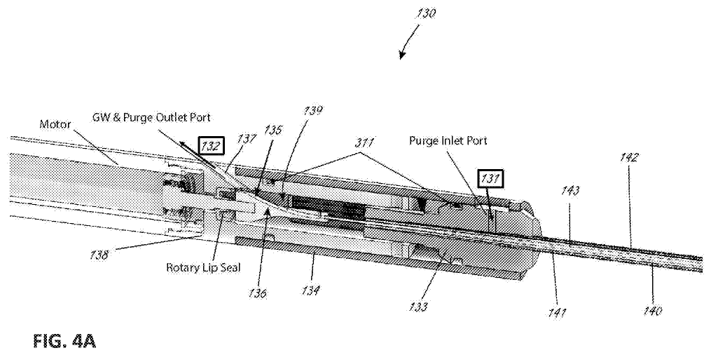

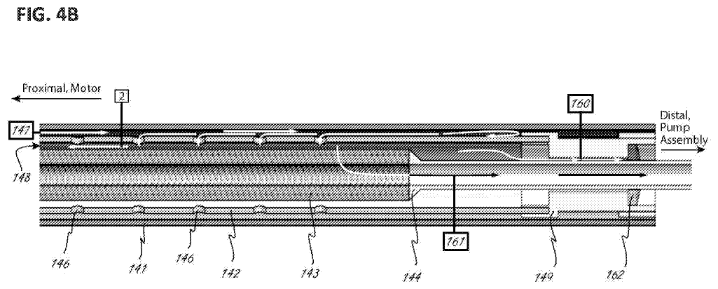

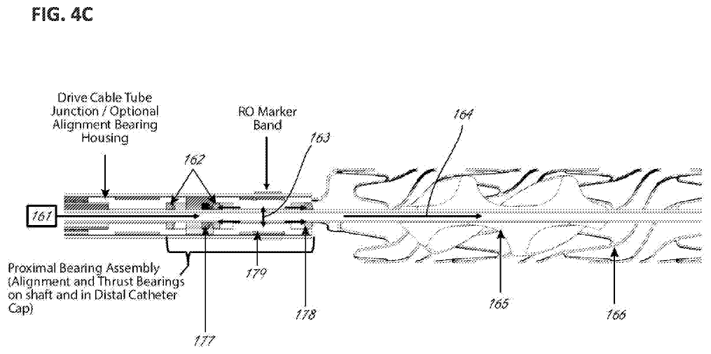

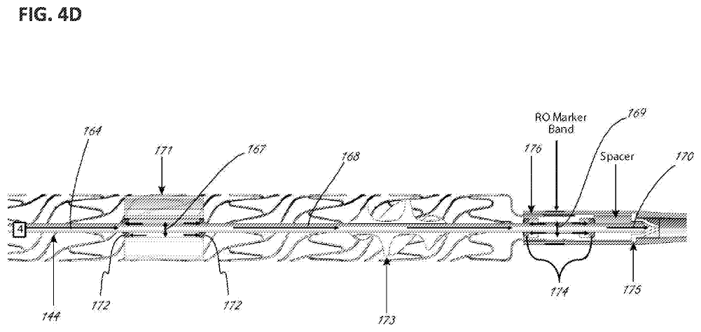

FIGS. 4A-4D illustrate an exemplary embodiment of a fluid delivery system incorporated into an exemplary fluid pump (e.g., blood pump) with a fluid inlet port and a fluid outlet port. FIG. 4A illustrates a portion of the device that is proximal to the one or more impellers, and in this embodiment includes a proximal end of a catheter, a motor assembly that causes the rotation of a drive cable and impeller(s), a fluid inlet port, and fluid outlet port, and a guidewire port that allows access to a guidewire pathway or lumen.

FIG. 4B shows a region of the device that is distal to the region shown in FIG. 4A, but includes some of the catheter components that are shown in FIG. 4A. FIG. 4C shows a region of the device distal to the region in FIG. 4B, and FIG. 4D shows a region of the device distal to the view in FIG. 15C.

While FIGS. 4A-4D illustrate different sections of an exemplary blood pumping device, it is understood that in alternative embodiments aspects of the system can vary. For example, in alternative embodiments the portion of the device with the impellers can vary and could only include a single impeller, or the expandable housing around the impeller could have a wide variety of configurations. It is understood that individual regions of the device can be incorporated by themselves into a variety of different types of blood pumps.

One aspect of this exemplary embodiment includes a guidewire access port that also functions as a fluid port, and in this embodiment a fluid outlet port. A motor sealing cap 138 includes, formed therein, a guidewire channel 137, including a guidewire port in a radially side surface that provides access from outside the device to channel 137. The motor sealing cap may be an optional component, and the guidewire channel 137 can alternatively be formed in a different part of the device (e.g., which may not function as a motor sealing cap). The device also includes drive cable coupler 135, which includes formed therein a guidewire channel 136, which is a portion of a guidewire pathway. Drive cable coupler 135 is rotated by the motor, and causes the rotation of drive cable 143, which causes rotation of the one or more impellers in the pump portion. These components are thus considered to be in rotational communication. Channel 137, including the guidewire port, is formed in the device and is not adapted to rotate when the motor rotates. Channel 136 formed in drive cable coupler 135 rotates when the drive cable coupler rotates. When drive cable coupler 135 is in the position shown in FIG. 4A, channel 137 is in alignment with channel 136, which allows a guidewire to be advanced through or removed from channel 137 and through channel 136. If the guidewire is being inserted, the guidewire can then be advanced further distally through the entire device and out a distal end, described in more detail below. As is also described in more detail below, the guidewire access port also acts as a fluid outlet port that allows return fluid to flow from return area 139 out of the outlet port.

One of the advantages of having the guidewire access port (part of channel 137) in the location that it is in this embodiment, is that, if needed after the pump portion has already been advanced to a location within the patient, a guidewire can be reinserted into the port and inserted all the way to and out of the distal end. Importantly, the guidewire can be reinserted without having to remove most of the device from the patient like with some rapid exchange designs, and without having to remove the motor assembly. This exemplary embodiment thus allows easy reentry of a guidewire without having to remove the motor assembly, and without having to remove the device from the subject.

Being able to reinsert the guidewire during use can be advantageous because it can, for example without limitation, allow for repositioning of the pump portion if desired or needed. For example, if the pump portion moves out of position relative to an anatomical landmark (e.g., an aortic valve), a guidewire may need to be inserted to safely reposition it relative to the anatomical landmark.

Because the guidewire path extends through a rotational component (e.g., drive cable coupler 135), it is important that the guidewire not be present in the guidewire path when the rotating component is active. The apparatuses herein can also include an automated sensing mechanism to detect the presence of the guidewire in the guidewire pathway, and/or a prevention mechanism that prevents the motor from being activated if the guidewire is in the lumen. For example without limitation, there could be a sensor that can selectively detect the presence of the guidewire in the guidewire pathway, and communicate that to a controller that prevents the motor from being activated.

In this embodiment there is a single fluid inlet channel or lumen 131 into which fluid can be delivered into the device. FIG. 4B illustrates a region of the device and illustrates different pathways the fluid can take after it has been delivered into the device. After the fluid is advanced into fluid inlet port channel 131 (which includes an inlet port), it travels through a space 147 between clean purge tube 141 and drive cable tube 142. This is considered clean input fluid. This pathway dead ends at distal catheter cap 149. The fluid passes through the one or more apertures 146 formed in a distal region of drive cable tube 142 as shown in FIG. 4B, entering into an annular space between drive cable tube 142 and drive cable 143. Some of this fluid (optionally most of the fluid) returns in the proximal direction through this annular space, lubricating and cooling drive cable 143 and flushing potential particulate along its path. This return fluid continues to flow proximally and into area 139 shown in FIG. 4A, and continues to flow through channel 137 and out of the fluid port (which is also the guidewire access port). A fluid outlet port thus also functions as a guidewire access port in this embodiment.

While most of the fluid returns proximally to area 139, some of the fluid, after it passes through apertures 146, continues distally beyond the distal end of the drive cable 143. Some of the fluid follows proximal bearing path 160 through alignment bearing 162 to prevent blood ingress. Fluid flow along path 160 to bearing 162 can be controlled by, for example, controlling input flow pressure and throttling of the return fluid at the proximal region of the device.

Some of the fluid, after passing through apertures 146, will flow through drive cable 143, along path 161, and will continue distally through the device (e.g., through hypotube 144) and out holes to lubricate any rotating surfaces and to prevent blood ingress, described in more detail below. Guidewire lumen 145 is thus positioned to also function as a distal bearing fluid flow path.

Some fluid flows distally along path 161, as shown in FIG. 4C, and passes through holes along path 163, to lubricate one or more of bearings 162, thrust bearing 177, and alignment bearing 178. Some of the fluid continues distally in the direction of arrow 164 shown in FIG. 4C, through impeller 165 (which in this embodiment is a proximal impeller). Some of the fluid passes through apertures along path 167 to lubricate optional alignment bearings 172 that support central member 171, which may be any of the collapsible support members, including any of the central or intermediate members herein. Some fluid continues distally through the guidewire lumen in the direction of arrow 168, through optional distal impeller 173. Some fluid passes through holes along path 169 to lubricate bearings 174 that are distal to the distal impeller. Some of the fluid may also flow through valve 175 and out the distal end of the device, helping prevent blood ingress.

In this exemplary embodiment a single flow path flowing through a tubular member (path 161 that extends distally through guidewire lumen shown in FIG. 4B) leads to (is in fluid communication with) at least three distally located bearing lubricating fluid paths, 163, 167, and 169, which lubricated three axially spaced bearing regions. In some alternative embodiments, there may be a single bearing region that is lubricated, two bearing regions that are lubricated, or even more than three bearings regions that are lubricated, depending on the number of structures disposed within the expandable housing that require bearings and thus lubrication.

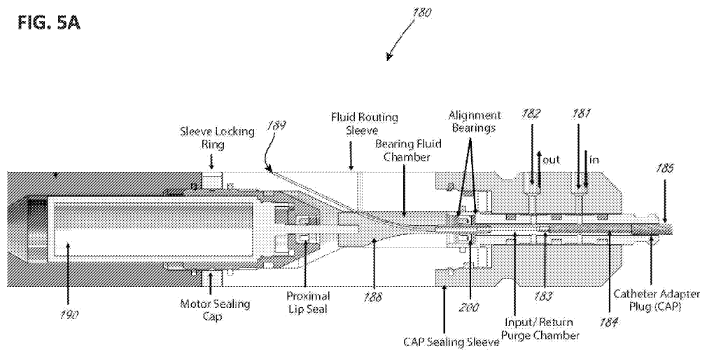

An exemplary method of using the device in FIGS. 4A-D includes inserting a guidewire near a target location (e.g., into a left ventricle via femoral artery access), then feeding the distal guidewire port over the guidewire and advancing the device over the guidewire towards the target location (e.g., an aortic valve). The method can also include removing the guidewire from the guidewire path, and coupling the proximal portion shown in FIG. 4A to a fluid inlet coupler and a fluid outlet coupler at the inlet and the outlet fluid locations, respectively. The motor can be activated to activate the one or more impellers. If the guidewire needed to be reinserted, the fluid out connector can be removed and a guidewire can be reinserted (e.g., for repositioning). The guidewire can then be removed and the fluid outlet coupler can again be put into fluid communication with the guidewire pathway. These methods or any of them individually can be incorporated into the use of any of the suitable devices herein, such as the device in FIGS. 5A and 5B. Additionally, any of the steps in any of the other exemplary methods of use herein, such as those below, may be incorporated into a use of the blood pump in this embodiment.

FIGS. 5A and 5B illustrate an exemplary embodiment of a fluid delivery system incorporated into an exemplary fluid pump (e.g., blood pump) with a first flow path with a first fluid inlet port and a first fluid outlet port. In this embodiment, however, there is also a second fluid flow path that is not in fluid communication with the first flow path. The device 180 in FIGS. 5A and 5B is similar to that shown in the embodiment in FIGS. 4A-D, except in this embodiment the fluid path 161 from FIG. 4B does not originate as fluid that flows through the drive cable. In this embodiment the fluid flow path that includes the guidewire lumen (see fluid path 196 in FIG. 5B) is in fluid communication with a separate and second fluid inlet port 189, which is also located to function as a guidewire access port, as shown in FIG. 5A. Drive cable 183 has a drive cable liner 187 on its inner surface to seal off the distal bearing flow path 196 (through the guidewire lumen). In this embodiment the guidewire access port does not function as a fluid outlet, like in FIGS. 4A-D, but as a fluid inlet port, and thus still functions as a fluid port or fluid access.

The blood pump also includes a first fluid path that includes inlet port 181 and outlet port 182 as shown in FIG. 5A. This flow path is very similar to the path in FIGS. 4A-D, except that it does not include the path through the drive cable and hypotube (i.e., does not include the guidewire lumen). The fluid is advanced through port inlet port 181, flows distally along path 197 in FIG. 5B, which is between clean purge tube 185 and drive cable tube 184. This path dead ends at a distal catheter cap, just as in the embodiment in FIGS. 4A-D. The fluid flows through holes in drive cable tube 184, and returns proximally in the annular space between drive cable tube 184 and drive cable 183. In this part of the path the fluid lubricates and cools the drive cable and flushes potential particulate along its path, carrying them proximally to fluid exit port 182 shown in FIG. 5A. Seal 200 prevents fluid from passing proximally to seal.

Fluid flowing through the first fluid path thus lubricates and cools the drive cable, as well as flushes potential particulates and returns to exit port 182. Fluid flowing through the second fluid path travels further distally through the system, and lubricates one or more distal bearings, just as in the embodiment in FIGS. 4A-D. For example, path 199 shown in FIG. 5B is the same as path 163 in FIG. 4C, which lubricates bearings in that bearing region. While not shown, the fluid flow path distal to the view shown in FIG. 5B can be exactly the same as in FIG. 4D, thus lubricating additional bearings, and optionally exiting through a valve at a distal end of the device. This second flow path can thus also prevent ingress of blood, which is described more fully in FIGS. 4A-D.

In any of the devices herein, the pump portion can include a distal end valve distal to the impeller to seal off the distal guidewire port after the guidewire is removed, but allows for guidewire reinserting therethrough.

The following disclosure provides exemplary method steps that may be performed when using any of the blood pumps, or portions thereof, described herein. It is understood that not all of the steps need to be performed, but rather the steps are intended to be an illustrative procedure. It is also intended that, if suitable, in some instances the order of one or more steps may be different.

Before use, the blood pump can be prepared for use by priming the lumens (including any annular spaces) and pump assembly with sterile solution (e.g., heparinized saline) to remove any air bubbles from any fluid lines. The catheter, including any number of purge lines, may then be connected to a console. Alternatively, the catheter may be connected to a console and/or a separate pump that are used to prime the catheter to remove air bubbles.

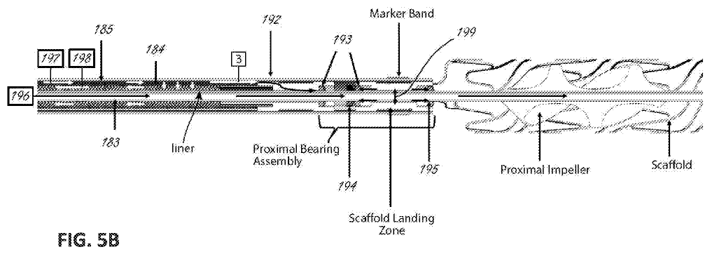

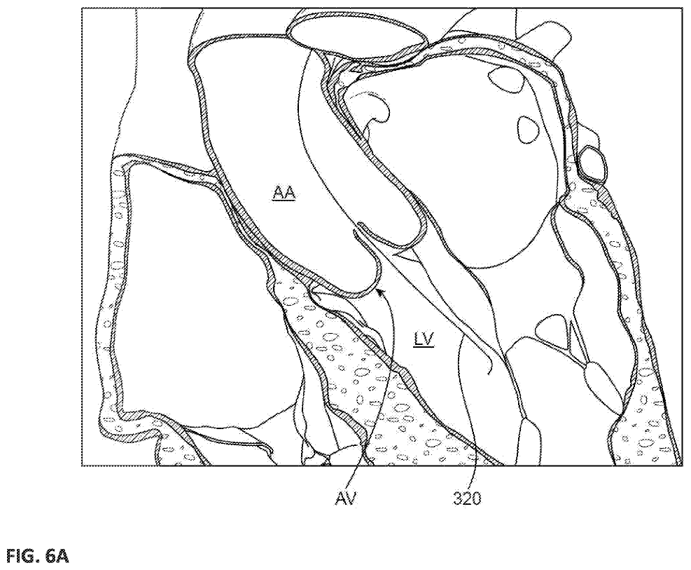

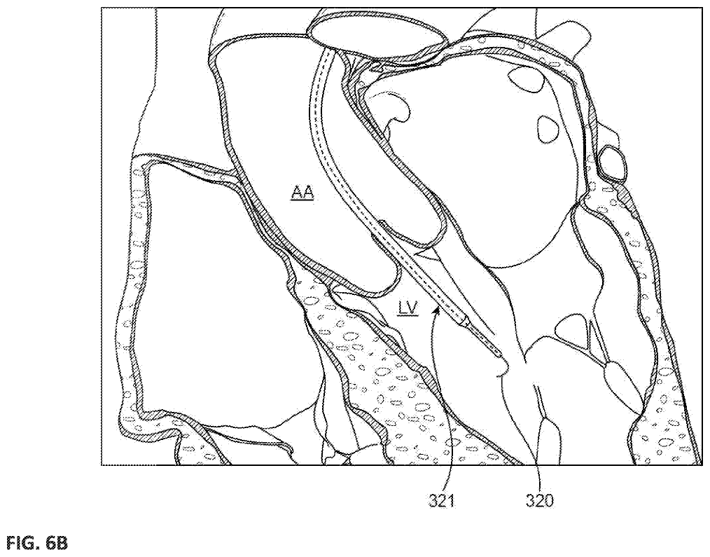

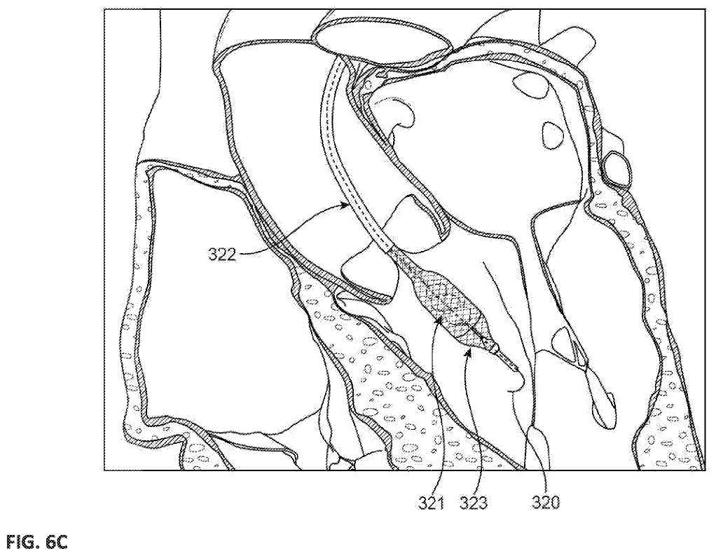

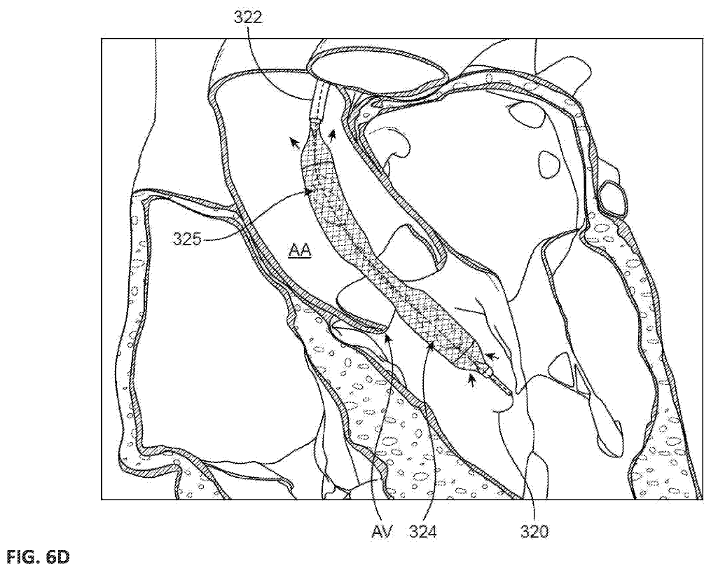

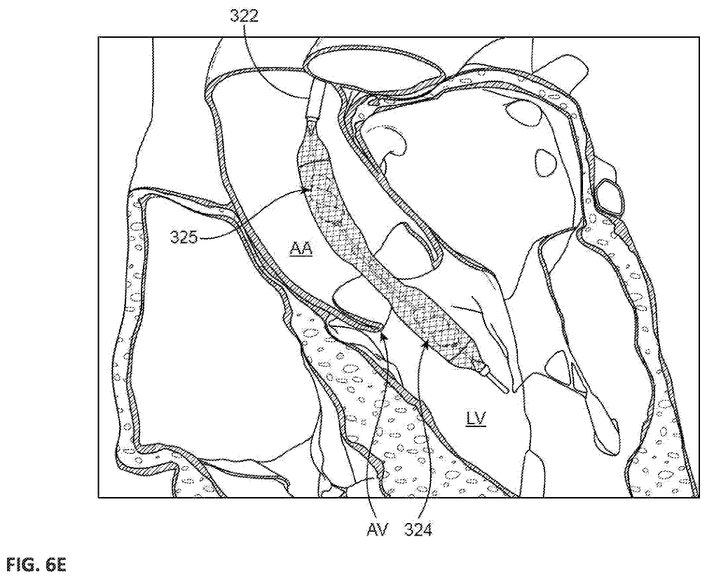

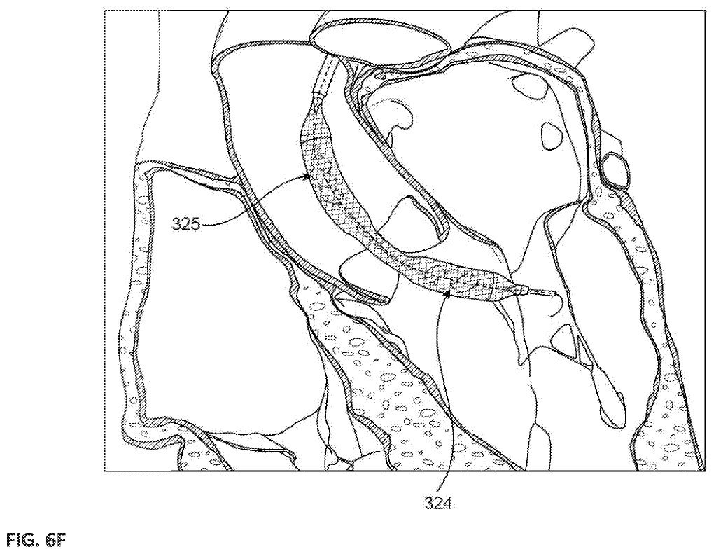

After priming the catheter, access to the patient's vasculature can be obtained (e.g., without limitation, via femoral access) using an appropriately sized introducer sheath. Using standard valve crossing techniques, a diagnostic pigtail catheter may then be advanced over a, for example, 0.035″ guide wire until the pigtail catheter is positioned securely in the target location (e.g., left ventricle). The guidewire can then be removed and a second wire 320 (e.g., a 0.018″ wire) can be inserted through the pigtail catheter. The pigtail catheter can then be removed (see FIG. 6A), and the blood pump 321 (including a catheter, catheter sheath, and pump portion within the sheath; see FIG. 6B) can be advanced over the second wire towards a target location, such as spanning an aortic valve “AV,” and into a target location (e.g., left ventricle “LV”), using, for example, one or more radiopaque markers to position the blood pump.

Once proper placement is confirmed, the catheter sheath 322 (see FIG. 6C) can be retracted, exposing first a distal region of the pump portion. In FIG. 6C a distal region of an expandable housing has been released from sheath 322 and is expanded, as is distal impeller 324. A proximal end of housing 323 and a proximal impeller are not yet released from sheath 322. Continued retraction of sheath 322 beyond the proximal end of housing 323 allows the housing 323 and proximal impeller 325 to expand (see FIG. 6D). The inflow region (shown with arrows even though the impellers are not yet rotating) and the distal impeller are in the left ventricle. The outflow (shown with arrows even though the impellers are not rotating yet) and proximal impeller are in the ascending aorta AA. The region of the outer housing in between the two impellers, which may be more flexible than the housing regions surrounding the impellers, as described in more detail herein, spans the aortic valve AV. In an exemplary operating position as shown, an inlet portion of the pump portion will be distal to the aortic valve, in the left ventricle, and an outlet of the pump portion will be proximal to the aortic valve, in the ascending aorta (“AA”).

The second wire (e.g., an 0.018″ guidewire) may then be moved prior to operation of the pump assembly (see FIG. 6E). If desired or needed, the pump portion can be deflected (active or passively) at one or more locations as described herein, as illustrated in FIG. 6F. For example, a region between two impellers can be deflected by tensioning a tensioning member that extends to a location between two impellers. The deflection may be desired or needed to accommodate the specific anatomy. As needed, the pump portion can be repositioned to achieve the intended placement, such as, for example, having a first impeller on one side of a heart valve and a second impeller on a second side of the heart valve. It is understood that in FIG. 6F, the pump portion is not in any way interfering or interacting with the mitral valve, even if it may appear that way from the figure.

Any number of purge lines may then be attached to the proximal portion of the blood pump that is disposed outside of the patient. For example, fluid inlet(s) lines and fluid outlet(s) lines may be attached to one or more fluid ports on the proximal portion of the blood pump. A purge process can then be initiated to move fluid into the blood pump through at least one fluid pathway. One or more Confirmation steps can be performed to confirm the purge is operating as intended before turning on the pump. The pump assembly can then be operated, causing rotation of the one or more impellers. Any one of flow rate(s), pressure(s), and motor operation can be monitored at any time.

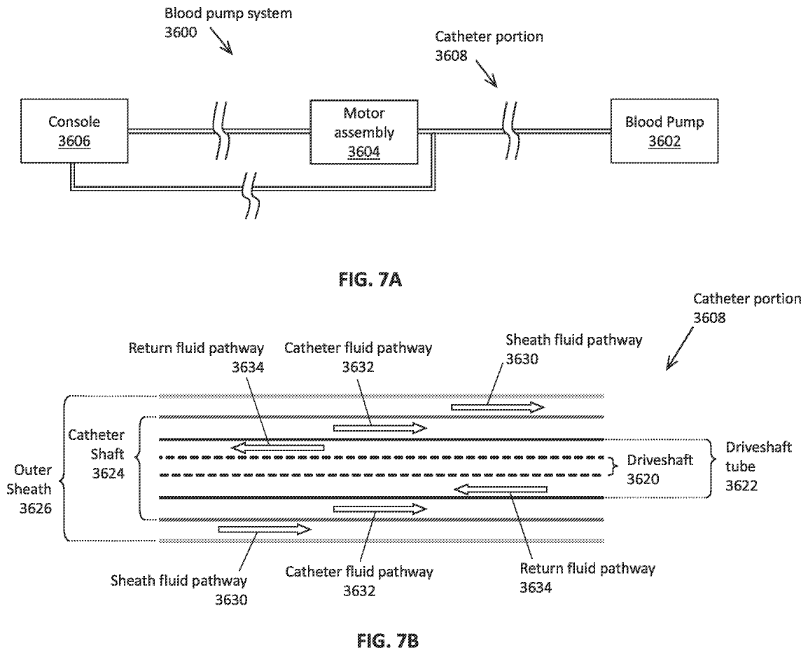

FIG. 7A schematically illustrates at least a portion of an exemplary blood pump system 3600. Any of the blood pump systems described herein can have the arrangement of system 3600. The exemplary blood pump system 3600 may include an external console 3606, a motor assembly 3604 and catheter portion 3608. A distal end of the catheter portion 3608 can include an intravascular blood pump 3602. The intravascular blood pump 3602 can be configured to enter a patient's vascular system to pump blood within the patient's body while the console 3606 and motor assembly 3604 remain outside of the patient's body. The console 3606 may include one or more controllers (e.g., as part of a computer) for controlling aspects of the motor assembly 3604 and blood pump 3602. The console 3606 may include a user interface (e.g., computer display) for interacting with a user. Console 3606 may be in fluid and/or electrical communication with the motor assembly 3604 via, for example, one or more electrical wires and/or one or more fluidic channels. In some examples, the console 3606 may be in fluid and/or electrical communication with the catheter portion 3608 via, for example, one or more electrical wires and/or one or more fluidic channels (e.g., bypassing the motor assembly 3604). The motor assembly 3604 may include one or more motors that are in rotational communication with one or more internal driveshafts (also referred to herein as drive cables) of the blood pump 3602. Any driveshaft and/or drive cable herein may be considered part of a rotational drive mechanism that communicates rotation from a motor to one or more impellers of the blood pump. In some cases, the motor assembly 3604 is part of a handle for controlling the blood pump 3602. In other cases, the motor assembly 3604 is a separate structural entity from a handle.

FIG. 7B schematically illustrates a close-up section view of at least a portion of an exemplary catheter portion 3608, and illustrates exemplary and optional fluid pathways therein. The catheter portion 3608 can include a series of coaxial tubular components including a hollow driveshaft 3620, a driveshaft tube 3622, a catheter shaft 3624 and an outer sheath 3626. The hollow driveshaft 3620 can be in rotational communication with one or more impellers of a pump portion of the blood pump 3602. The hollow driveshaft 3620 can be configured to rotate by being rotatably coupled to a motor of the motor assembly 3604. The hollow driveshaft 3620 may include an inner lumen to accommodate, for example, a guidewire. In some cases, at least a portion of the hollow driveshaft 3620 may include a porous material through which some fluid (e.g., saline, dextrose, return fluid, etc.) may penetrate through the walls of the driveshaft 3620 and into the inner lumen of the driveshaft 3620. In some examples, the driveshaft 3620 walls may include a mesh material. The driveshaft tube 3622 may be configured to house the rotatable driveshaft 3620. The catheter shaft 3624 (which may be referred to simply as a catheter, and which may include one or more layers of material) may house components of the blood pump 3602 at a distal portion of the catheter 3624, such as one or more bearing assemblies of the blood pump 3602. The outer sheath 3626 may house some or all of the pump portion of the blood pump 3602 within a distal portion of the sheath 3626, for example, during delivery of the blood pump 3602 to a target location within the patient. Once, in position, the sheath 3626 may be retracted to allow expansion of the expandable portions of the blood pump 3602. In some cases, the motor assembly 3604 may be adapted to rotate the driveshaft 3620 and not be adapted to rotate the driveshaft tube 3622, catheter shaft 3624 and/or sheath 366.

Depending on the particular design of the catheter portion, the catheter portion may include one or more fluid pathways that allows fluid to flow in the annular spaces between each of the components of the catheter portion 3608. For example, clean fluid (e.g., clean saline) may flow (e.g., by being pumped with a pump) toward the blood pump 3602 via a sheath fluid pathway 3630 between the sheath 3626 and the catheter shaft 3624. Fluid flow through the sheath fluid pathway 3630 may prevent blood from stagnating and forming clots in the annular space between the sheath 3626 and the catheter shaft 3624 at a distal end of the sheath 3626. Fluid from the sheath fluid pathway 3630 may enter the patient's body with no substantial return fluid pathway. Clean fluid (e.g., saline pumped from the saline bag in the console) may also flow (e.g., by being pumped) toward the blood pump 3602 via a catheter fluid pathway 3632 between the catheter shaft 3624 and the driveshaft tube 3622. Some or all of the fluid in the catheter fluid pathway 3632 may return from the blood pump 3602 via a return fluid pathway 3634 (which may be referred to in any embodiment herein as a waste fluid pathway). Flowing fluid through the catheter fluid pathway 3632 and return fluid pathway 3634 may cool and/or lubricate moving components (e.g., the rotating driveshaft 3620 and bearings) within the blood pump 3602. The catheter fluid pathway 3632 and return fluid pathway 3634 may flush and keep possible debris (e.g., from the moving components) from entering the patient's body. In some examples, where the walls of the driveshaft 3620 has some porosity, fluid within the return fluid pathway 3634 may enter the inner lumen of the driveshaft 3620.

Optionally, clean fluid for the sheath fluid pathway 3630 and the catheter fluid pathway 3632 may be provided by a console 3606, which may include one or more clean fluid sources (e.g., saline bags) and a pump assembly (e.g., peristaltic pump assembly) for pushing clean fluid toward the blood pump 3602. In some examples, the clean fluid may be provided through a catheter fluid inlet and a sheath fluid inlet between the motor assembly 3604 and the blood pump 3602. In some cases, one or both of the catheter fluid inlet and the sheath fluid inlet are part of (or connected to) the motor assembly 3604. In some examples, the return fluid pathway 3634 may flow through the motor assembly 3604 and toward a waste reservoir, which optionally may be connected to (or part of) such as by being secured to, the console 3606.

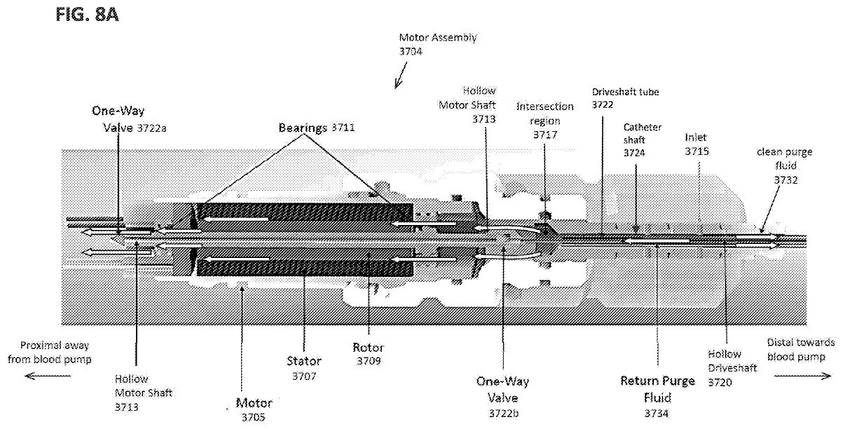

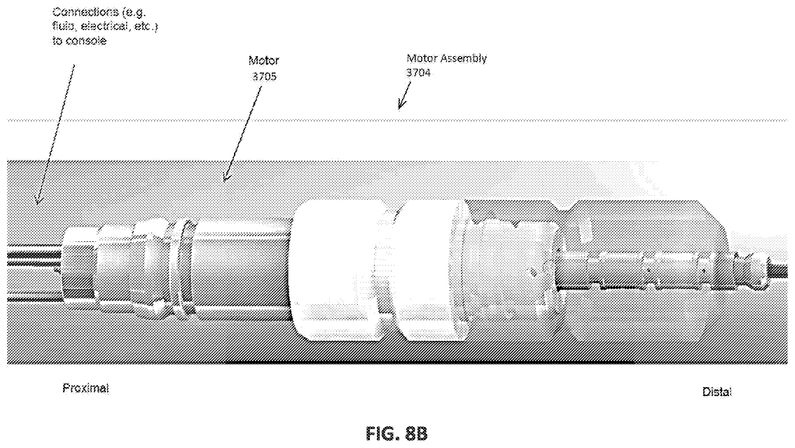

In some examples, the motor assembly 3604 is configured to allow fluid to pass therethrough to cool, lubricate and/or flush various internal components of the motor assembly 3604, as well as optionally providing a pathway for at least some of the return fluid through the system. FIGS. 8A and 8B illustrate an exemplary motor assembly 3704 showing an exemplary fluid pathway therethrough. Clean fluid (e.g., from the console) can enter an inlet 3715 that is, in this case, at a distal portion of the motor assembly 3704. The clean purge fluid 3732 can travel in a distal direction toward the blood pump in the annular space between the catheter shaft 3724 and the driveshaft tube 3722. The driveshaft 3720 may be rotationally coupled with one or more impellers of the blood pump. In some examples, the clean purge fluid 3732 pushed toward the blood pump forms the catheter fluid pathway for cooling, lubricating and/or flushing one or more components of the blood pump, such as the driveshaft and/or one or more bearings in the pump section, such as any of the pump sections incorporated by reference herein.

At least some (e.g., nominally all) of the clean purge fluid 3732 returns from the blood pump as return purge fluid 3734 through the driveshaft tube 3722. The hollow driveshaft 3720 may be at least partially permeable to fluid such that some of the fluid within the driveshaft tube 3722 seeps into the inner lumen of the hollow driveshaft 3720. The return purge fluid 3734 can travel proximally through the driveshaft tube 37222 and exit an intersection region 3717.

From the intersection region 3717, the return fluid can be directed in an annular space around a hollow motor shaft 3713 that is rotationally coupled to the hollow driveshaft 3720. The return purge fluid can then be directed through spaces between rotational elements (e.g., balls) of a first bearing and into an annular space between a stator 3707 and a rotor 3709 of a motor 3705. The motor 3705 can be configured to rotate the hollow motor shaft 3713, which is rotationally coupled to the hollow driveshaft 3720. Moving further proximally, the return purge fluid can exit the motor 3705 through spaces between rotational elements (e.g., balls) of a second bearing and exit a proximal end of the motor assembly 3704. Once exited the motor assembly 3704, the return fluid may be directed to a waste reservoir, for example, at an external console of the blood pump system.

In some examples, the motor assembly 3704 optionally includes one or more one-way valves (e.g., 3722a and 3722b), which can prevent fluid from entering the hollow motor shaft 3713 within the motor 3705. This may keep that lumen of the hollow motor shaft 3713 clean in the event a guide wire needs to be advanced distally back through the blood pump through this lumen.

An exemplary benefit of the configuration shown in FIGS. 8A and 8B is that the return purge fluid 3734 running through the motor 3705 can keep the motor bearings 3711 lubricated. The return purge fluid 3734 may flush out debris in the bearings 3711. In some examples, rotary seals are not necessary since the motor 3705 can have sealed electronics and can function normally while filled with purge fluid. In this example, the return fluid path through the motor 3705 may provide some degree of motor cooling, although creating a pathway for return fluid to flow out of the blood pump system may be a more important design feature.

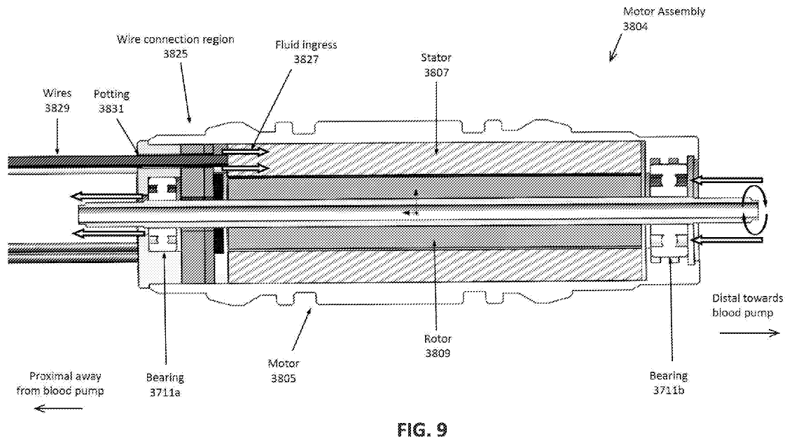

FIG. 9 shows an exemplary motor assembly 3804 of an intravascular blood pump, illustrating a possible fluid ingress site to a stator 3807. In this example, the fluid may be able to enter the stator 3807 around a wire connection region 3825 of the stator 3807 where wires 3829 exit the stator 3807. For example, fluid may enter spaces between the wires 3829 and the housing surrounding the motor 3805 if potting 3831 does not provide a sufficient fluid impermeable seal. Another possible path of fluid ingress may be at junctions between parts of the housing surrounding the motor 3805, such as the exemplary circumferential weld junction 3833. If such weld regions are improperly formed, these regions may also allow fluid to enter the stator 3807. Fluid ingress into the stator 3807 may cause an electrical short and/or cause the stator 3807 to corrode.

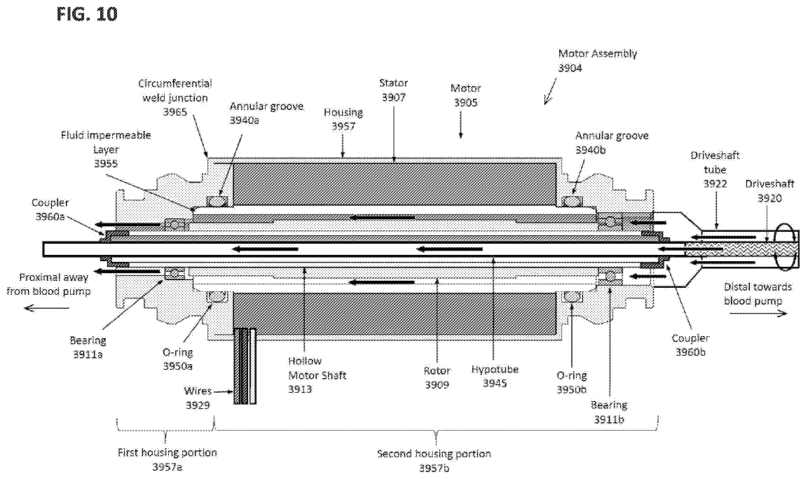

FIG. 10 illustrates an exemplary motor assembly 3904 configured to fluidically isolate the stator from the fluidic pathway, thereby addressing the fluid ingress problems described with respect to FIG. 9. In this example, a fluid impermeable layer 3955 is positioned between the stator 3907 and the rotor 3909. The fluid impermeable layer 3955 may act as a barrier to prevent fluid from the fluid pathway within the motor from reaching the stator 3907. The fluid impermeable layer 3955 may have a cylindrical shape with the rotor 3907 positioned within the inner lumen of the fluid impermeable layer 3955. The fluid impermeable layer 3955 may be made of a non-electrically conductive material to prevent interference with the functioning of the motor 3905. In some cases, the fluid impermeable layer 3955 comprises a polymer material. As shown, fluid (e.g., return purge fluid) may flow radially within the impermeable layer 3955 and radially outside of the rotor 3909.

The fluid impermeable layer 3955 may be secured in place within the motor assembly 3904. For example, the housing 3957 of the motor assembly 3904 may include multiple sections that are coupled (e.g., bonded, welded, or otherwise coupled) together with the fluid impermeable layer 3955 disposed therein. In the non-limiting example shown, the housing 3957 includes a first housing portion 3957a and a second housing portion 3957b that are coupled together by a circumferential weld 3965, thereby encasing the fluid impermeable layer 3955 within the housing 3957. In alternative examples, the housing may include other arrangements of a plurality of housing portions that are coupled together (at one or more coupling locations) to secure the fluid impermeable layer therein. For example, the housing may include more than two housing portions coupled together. Additionally, for example, first housing portion 3975a and second housing portion 3975b may be coupled (e.g., welded) in a distal portion of the motor assembly.

The motor assembly 3904 may include one or more sealing elements or members (e.g., one or more O-rings) strategically placed to prevent fluid from reaching the stator 3907. The exemplary motor assembly 3904 includes a first sealing element (e.g., O-ring in this example) 3950a proximally located with respect to the stator 3907 and a second O-ring 3950b distally located with respect to the stator 3907. These O-rings 3950a and 3950b positioned either side of the stator 3907 can be sized and positioned to prevent fluid from reaching the stator 3907 from axial directions. The O-rings 3950a and 3950b may be positioned within annular groves 3940a and 3940b of the motor assembly housing 3957. In some examples, the O-rings 3950a and 3950b are in contact with and form a seal with the fluid impermeable layer 3955.

In some examples, wires 3929 for the stator 3907 may be configured to extend radially outward from the stator 3907 so that their entry points into the stator 3907, which may be easy entry points for fluid ingress, are situated away from the fluid path. The wires 3929 may extend through a slot on an outer portion of the motor assembly housing 3957. In some examples, the wires 3929 enter a sealed handle compartment.

Example fluid pathways of return fluid from the blood pump through the exemplary motor assembly 3904 are shown in arrows in FIG. 10. As shown, the return fluid can enter from a distal side of the motor 3905 (relative to the console) via driveshaft tube 3922 that surrounds the rotatable driveshaft 3920. The driveshaft 3920 may be permeable to fluid such that some fluid within the driveshaft tube 3922 may enter within the inner lumen of the driveshaft 3920. The return fluid from the driveshaft tube 3922 and driveshaft 3920 travel two different fluid pathways through the motor 3905. In a first fluid pathway, fluid can travel though a distal bearing 3911b (e.g., to cool, lubricate and/or flush the distal bearing 3911b), in an annular space between the fluid impermeable layer 3955 and the rotor 3929 (e.g., to cool, lubricate and/or flush the rotor 3909 and/or stator 3907), through a proximal bearing 3911a (e.g., to cool, lubricate and/or flush the proximal bearing 3911a), and out a proximal end of the motor assembly 3904.

In a second fluid pathway through the motor 3905, fluid may travel through a hypotube 3945, which is rotatably coupled to the driveshaft 3920 (as shown), and out the proximal end of the motor assembly 3904. The hypotube 3945 can be positioned within the lumen of a hollow motor shaft 3913, which is rotatably coupled to the rotor 3909. The hypotube 3945 may be impermeable to fluid passage therethrough, thereby preventing fluid from entering in the annular space between the hypotube 3945 and the hollow motor shaft 3913. The hypotube 3945 may be rotatably coupled to the hollow motor shaft 3913 by couplers 3960a and 3960b, which may have annular shapes to accommodate the hypotube 3945 positioned therethrough. The hypotube 3945 may be configured to accommodate a guidewire therethrough. Return fluid exiting from the proximal side of the motor assembly 3904 can travel to a waste fluid line toward a waste fluid reservoir, for example.

Purge fluid as used herein may also be referred to as a lubricating fluid, flushing fluid and/or a cooling fluid, and vice versa.

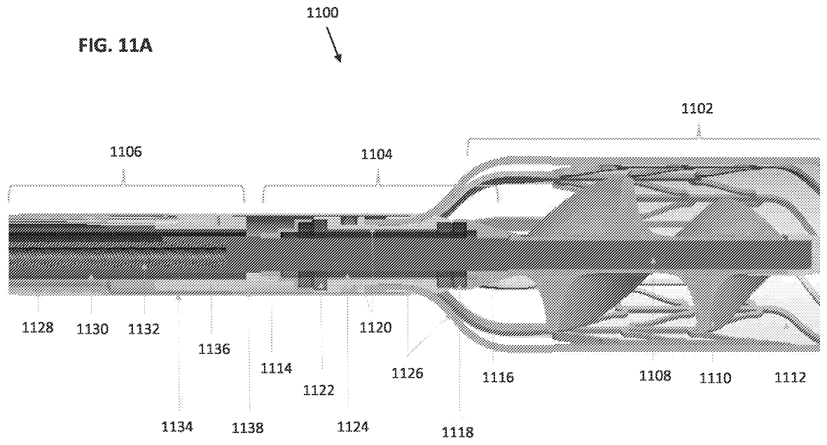

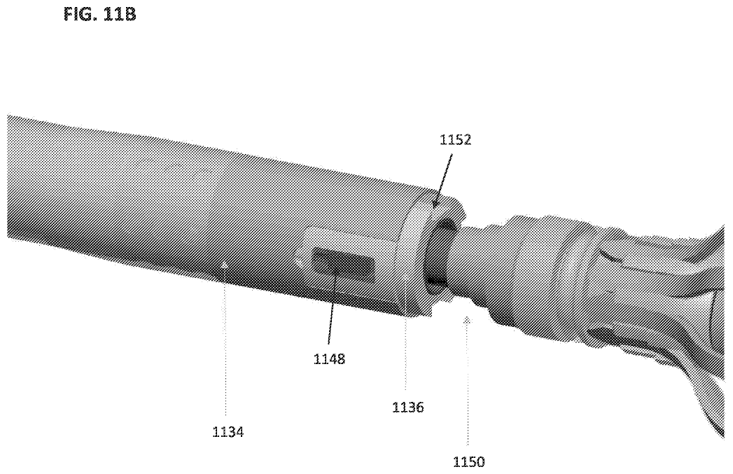

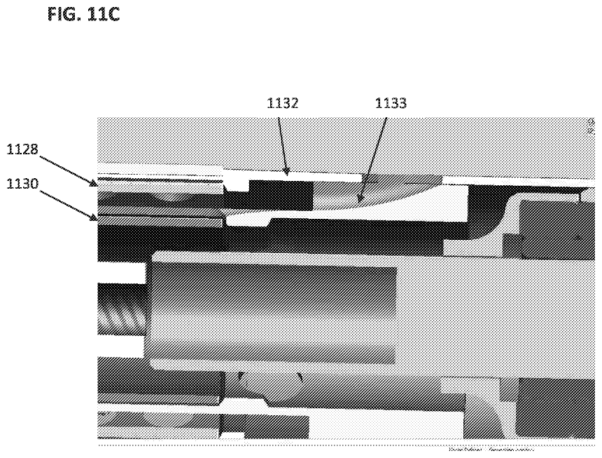

FIGS. 11A-11C illustrate another embodiment of a blood pump system 1100 that can include a pump portion 1102, a bearing assembly 1104, and a distal catheter portion 1106. Pump portion can include, for example, an impeller shaft 1108, a proximal impeller 1110, and an expandable scaffold or shroud 1112. The bearing assembly 1104 can include a proximal bearing housing 1114 and a distal bearing housing 1116. These bearing housings can be configured to rotate around the impeller shaft. Each of the bearing housings can include an outer bearing 1118 that also rotates around the impeller shaft. A central assembly 1120 can be fixed in place and can include a pair of thrust bearings 1122 on either side of the central assembly which are fixed in place, with a rotating central bearing 1124 between the thrust bearings in the central assembly 1120.

The distal catheter portion 1106 can comprise a clean purge tube 1128, a drive cable tube 1130 disposed in the clean purge tube, and a drive cable 1132 disposed in the drive cable tube. The distal catheter portion can further include a distal coupler 1134, a purge channel tube 1136 that includes a proximal sensor housing (described below), and a scaffold sleeve 1138 that is connected (e.g., welded) to the purge channel tube 1136 and the central assembly 1120. A flow path of purge fluid from outside the catheter can be delivered into the annular space between the drive cable tube and the clean purge tube. This purge fluid can pass from this annular space through the purge channel tube via fluid channels within the purge channel tube.

The pump portion 1102 can be joined to the bearing assembly 1104 with a plurality of struts 1126 (e.g., coupling of the struts to the central assembly 1120).

FIG. 11B is another view of the blood pump system 1100 including the distal coupler 1134, the purge channel tube 1136, and a proximal sensor 1148. Arrow 1150 shows where the scaffold sleeve (e.g., a scaffold sleeve 1138) would be positioned but is not illustrated in this figure to show additional details of the purge channel tube 1136, including flow channels 1152. It is noted that the scaffold sleeve configured to connect the purge channel tube 1136 to the central assembly, as described above.

FIG. 11C is a cross-sectional view of the distal coupler 1132. As shown, the distal coupler can serve to hold the drive cable tube 1130 and the clean purge tube 1128 in place, and to maintain the annular space between the clean purge tube and the drive cable tube. Additionally, the distal coupler 1132 can include a sensor wire ramp 1133 configured to route the sensor wire from the sensor (as shown in FIG. 11B) into the annular space between the drive cable tube 1130 and the clean purge tube 1128 (e.g., in the fluid channel between the drive cable tube and the clean purge tube).

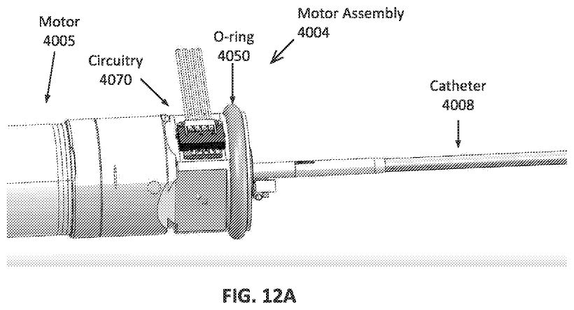

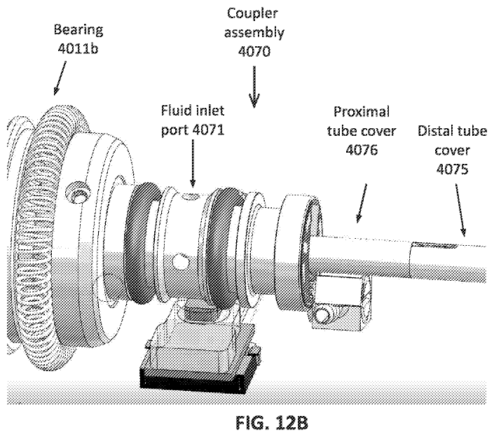

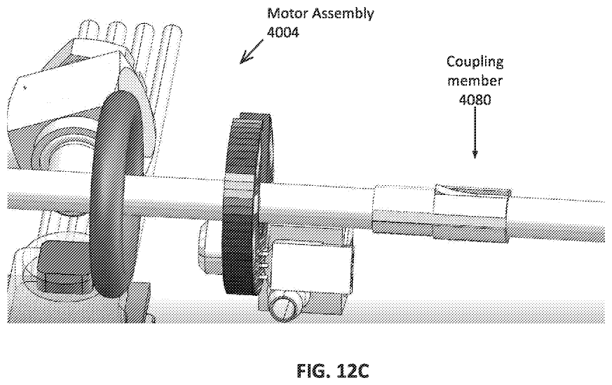

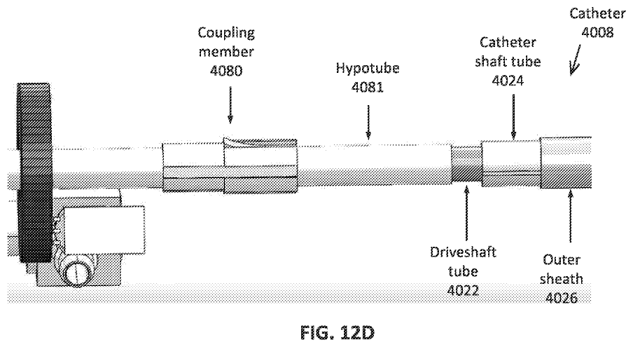

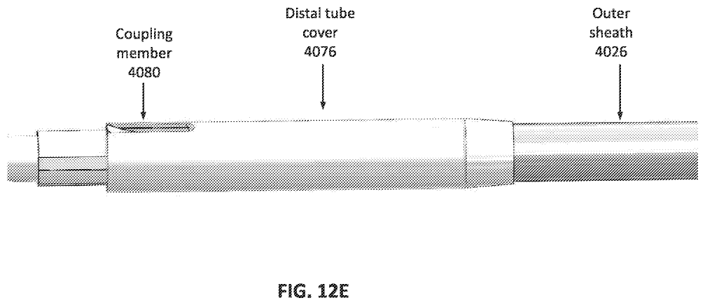

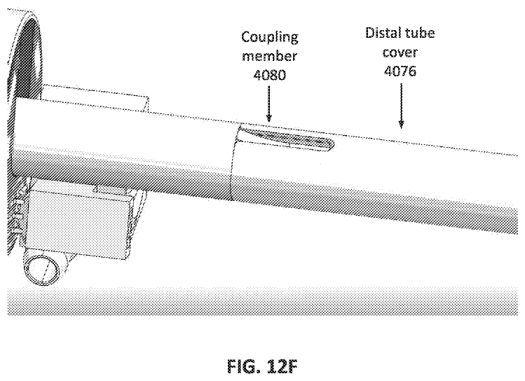

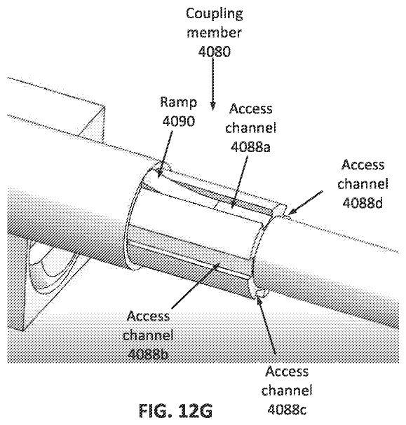

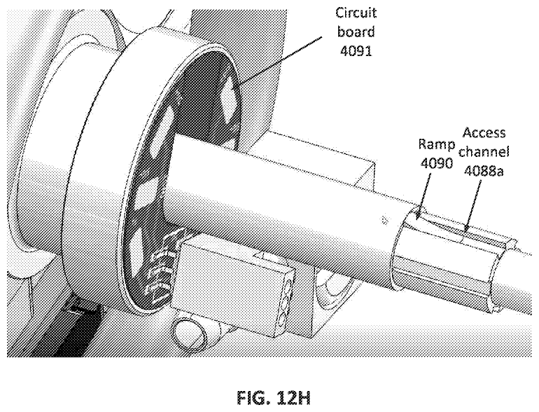

FIGS. 12A-12H illustrate a distal region of a motor assembly 4004 showing various fluid controlling features. As described above, the motor assembly 4004 may be part of a handle for controlling aspects of the catheter device. FIG. 12A shows a distal region of the motor assembly 4004 where the catheter 4008, which includes various layers of tubing pathways), enters the motor assembly 4004. FIG. 12B shows the distal region of the motor assembly 4004 where portions of the housing supporting circuitry 4070 and an o-ring 4050 are removed to reveal a coupler assembly 4070. In this example, at least a portion of the coupler assembly 4070 is situated distal to a distal bearing 4011b of the motor 4005. The coupler assembly may be is physically connected to the motor 4005 and configured to act together with the motor 4005 under compression and tension. The coupler assembly 4070 includes a fluid inlet port 4071 for routing clean fluid from the console (e.g., 3606) and distally toward the catheter 4008 (e.g., via catheter fluid pathway 3632 and/or sheath fluid pathway 3630 in FIG. 7B). The coupler assembly 4070 provides a means for connecting the fluid pathways and the driveshaft (e.g., 3620 in FIG. 7B) of the catheter 4008 to the motor 4005.

FIGS. 12C and 12D illustrate the distal region of the motor assembly 4004 with a proximal tube cover 4075, a distal tube cover 4076, and portions of the coupler assembly 4070 removed. As described above, the catheter 4008 includes the driveshaft tube 4022, catheter shaft 4024, and outer sheath 4026. A coupling member 4080 has a central lumen that accommodates a hypotube 4081 therein. In turn, the hypotube 4081 includes a driveshaft tube 4022 therein, where the driveshaft tube 4022 accommodates the rotating driveshaft therein. The coupling member 4080 may be fixedly coupled (e.g., welded) to the hypotube 4081, and the hypotube 4081 may be fixedly coupled (e.g., bonded) to the driveshaft tube 4022. In some examples, the driveshaft tube 4022 is made of a polymer (e.g., polyimide) material, and the hypotube 4081 is made of one or more metal materials.

FIGS. 12E-F show the distal tube cover 4076 positioned around a distal portion of the coupling member 4080, and FIG. 40F shows the proximal tube cover 4075 positioned around a proximal portion of the coupling member 4080. The proximal tube cover 4075 and the distal tube cover 4076 may be fixedly coupled (e.g., welded) to the outer surface of the coupling member 4080. In some examples, the proximal tube cover 4075 and distal tube cover 4076 are made of one or more metal materials. The coupling member 4080 may be fixedly coupled along any portion of the hypotube 4081 to axially align with proximal tube cover 4075 and distal tube cover 4076. In this way, the hypotube 4081 may serve to compensate for manufacturing tolerances related to axial length of the proximal tube cover 4075 and distal tube cover 4076.

FIG. 12G shows a closeup view of the coupling member 4080. The coupling member 4080 includes a number of access channels 4088a, 4088b, 4088c, 4088d, which are configured to provide access for fluid (e.g., saline) and/or elongate elements (e.g., one or more wires, one or more optical fibers, and/or one or more fluid or air tubes) to pass from/to the motor assembly 4004 and to/from the catheter 4008. In this example, the access channels correspond to longitudinal cutouts along the outer surface of the coupling member 4080, which provide space between the outer surface of the coupling member 4080 and the proximal tube cover 4075 and distal tube cover 4076 (See, FIG. 12F). Although in this example the coupling member 4080 includes five access channels (e.g., one additional access channel on opposite side of the coupling member 4080 in FIG. 12G), any number of access channels may be used. For example, the coupling member 4080 may include 1, 2, 3, 4, 5, 6, 7, 8 or more access channels.

In some cases, the access channels may be configured provide access for different components. For example, the access channels 4088b, 4088c, and 4088d may be configured to accommodate fluid (referred to as fluid channels), while the access channel 4088a may be configured to accommodate one or more elongate components (e.g., electrical wire(s) for sensor(s)), one or more optical fibers, and/or one or more air tubes). In some examples, the access channels 4088b, 4088c, and 4088d are configured to direct clean purge fluid from the coupler assembly 4070 to the catheter 4008. In this way, the fluid access channels 4088b, 4088c, and 4088d may provide a means for fluid to get distally past the coupling member 4080. In some examples, the access channel 4088a is configured to accommodate one or more wires for sensor(s) (e.g., temperature sensor(s), pressure sensor(s), etc. In the example shown in FIG. 12H, the access channel 4088a includes a ramp 4090 that is configured to direct one or more wires radially outward toward a circuit board 4091, where the wire(s) can be soldered to one or more circuit board components.

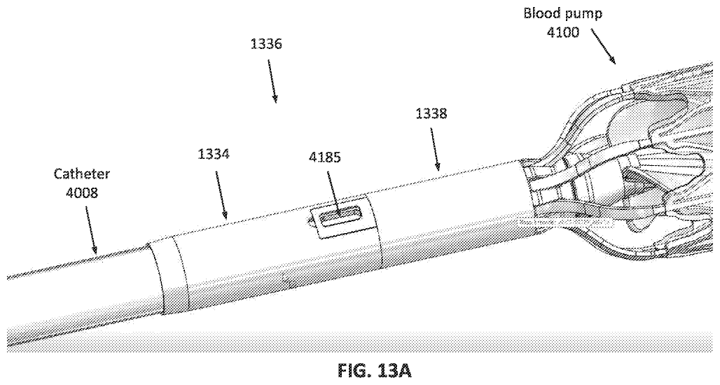

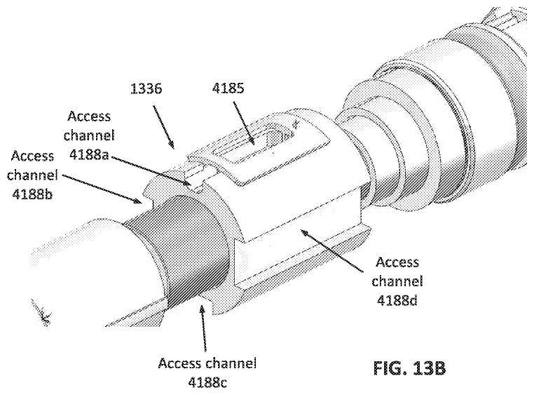

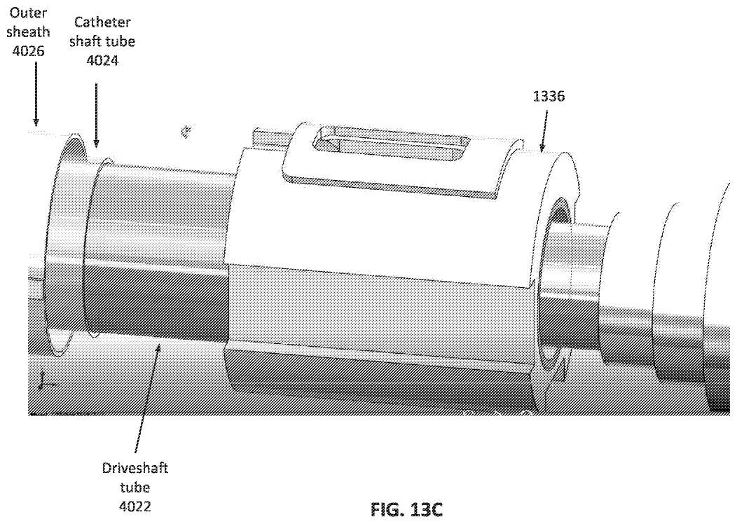

FIGS. 13A-13C illustrate a proximal portion of an exemplary blood pump 4100 showing additional views of a purge channel tube 1336, distal coupler 1334, scaffold sleeve 1338 near the blood pump 4100. As shown in FIG. 41A, the sensor holder 4185 can be located between the scaffold sleeve 1338 and the distal coupler, within the purge channel tube 1336.

FIG. 13B shows the distal coupler 1334 and the scaffold sleeve 1338 removed to reveal the purge channel tube 1336. The distal coupler and the scaffold sleeve may be fixedly coupled (e.g., welded) to the outer surface of the purge channel tube. The purge channel tube may include access channels 4188a, 4188b, 4188c, 4188d, which may correspond to longitudinal cutouts along the outer surface of the purge channel tube. Although in this example the purge channel tube includes four access channels, any number of access channels may be used. For example, the purge channel tube may include 1, 2, 3, 4, 5, 6, 7, 8 or more access channels.

In this example, the access channel 4188a includes the sensor holder 4185, which is sized and shaped to support one or more sensors (e.g., pressure sensor(s)) therein. In some examples, the sensor(s) may be fixedly coupled within the sensor holder 4185 using adhesive(s). As shown, the access channel 4188a includes an open proximal end so that one or more wires may extend proximally from the sensor holder 4185 and proximally out of the purge channel tube toward the motor assembly (e.g., 4004, FIGS. 12A-12H). Such wire(s) may be coupled to (e.g., laminated to) the outer surface of the driveshaft tube 4022, and thus be positioned between the driveshaft tube 4022 and the catheter shaft tube 4024. Thus, such wire(s) may be positioned between the driveshaft (within the driveshaft tube 4022) and fluid carried between the catheter shaft tube 4024 and the outer sheath 4026. The access channels 4188b, 4188c and 4188d may be configured to accommodate and direct fluid from/to the blood pump 4104 and to/from the catheter 4008.

FIG. 13C shows how the purge channel tube 1336 is positioned around the driveshaft tube 4022. The purge channel tube may be fixedly coupled (e.g., bonded) to the driveshaft tube 4022. A distal end of the purge channel tube may be aligned with a distal end of the driveshaft tube 4022.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A blood pump system, comprising:
a catheter having a distal end coupled to a blood pump, the blood pump having a driveshaft rotationally coupled to one or more impellers of the blood pump; and
a motor assembly having a distal portion coupled to the catheter, the motor assembly including a motor rotationally coupled to the driveshaft, wherein the distal portion of the motor assembly includes a coupling member comprising:
one or more fluid channels configured to direct fluid from the motor assembly toward the blood pump via the catheter; and
one or more access channels each configured to direct one or more elongate components from the blood pump toward the motor assembly via the catheter.

2. The blood pump system of claim 1, wherein the one or more elongate components includes one or more of: one or more electrical wires, one or more optical fibers, and one or more air tubes.

3. The blood pump system of claim 1, wherein the one or more fluid channels and the one or more access channels are longitudinal cutouts along an outer surface of the coupling member.

4. The blood pump system of claim 1, further comprising one or more tube covers fixedly coupled to an outer surface of the coupling member, wherein the one or more fluid channels and the one or more access channels provide spaces between the one or more tube covers and the coupling member.

5. The blood pump system of claim 1, wherein the catheter includes a drive shaft tube, a catheter shaft tube, and an outer sheath, wherein the drive shaft tube is positioned radially within the catheter shaft tube, and the catheter shaft tube is positioned radially within the outer sheath.

6. The blood pump system of claim 5, wherein the one or more fluid channels is configured to direct fluid between the catheter shaft tube and the outer sheath, and the one or more access channels is configured to direct the one or more elongate components between the drive shaft tube and the catheter shaft tube.

7. The blood pump system of claim 1, wherein the coupling member is fixedly coupled to a hypotube that surrounds a driveshaft tube, the driveshaft tube accommodating the driveshaft therein.

8. The blood pump system of claim 1, wherein the distal portion of the motor assembly further includes a coupler assembly, the coupler assembly including a fluid inlet port configured to direct fluid from an external console of the blood pump system toward the coupling member.

9. The blood pump system of claim 1, further comprising a second coupling member proximal to the blood pump, the second coupling member including:
one or more one or more second fluid channels configured to direct fluid from the catheter toward the blood pump; and
one or more second access channels each configured to direct one or more elongate components from the blood pump toward the catheter.

10. The blood pump system of claim 9, wherein the second one or more access channels include a sensor holder configured to support one or more sensors therein.

11. The blood pump system of claim 10, wherein the one or more sensors includes a pressure sensor.

12. A blood pump system, comprising:
a motor assembly including a motor rotationally coupled to a driveshaft;
a catheter including proximal portion coupled to the motor assembly, the catheter including a distal portion coupled to a blood pump, the blood pump having one or more impellers rotationally coupled to the driveshaft, wherein the distal portion of the catheter includes a coupling member comprising:
one or more fluid channels configured to direct fluid from the motor assembly toward the blood pump via the catheter; and
one or more access channels each configured to direct one or more elongate components from the blood pump toward the motor assembly via the catheter.

13. The blood pump system of claim 12, wherein the one or more elongate components includes one or more of: one or more electrical wires, one or more optical fibers, and one or more air tubes.

14. The blood pump system of claim 12, wherein the one or more access channels includes a sensor holder configured to support one or more sensors therein.

15. The blood pump system of claim 14, wherein the one or more sensors includes a pressure sensor.

16. The blood pump system of claim 12, wherein the one or more fluid channels and the one or more access channels are longitudinal cutouts along an outer surface of the coupling member.

17. The blood pump system of claim 12, further comprising one or more tube covers fixedly coupled to an outer surface of the coupling member, wherein the one or more fluid channels and the one or more access channels provide spaces between the one or more tube covers and the coupling member.

18. The blood pump system of claim 12, wherein the catheter includes a drive shaft tube, a catheter shaft tube, and an outer sheath, wherein the drive shaft tube is positioned radially within the catheter shaft tube, and the catheter shaft tube is positioned radially within the outer sheath.

19. The blood pump system of claim 18, wherein the one or more fluid channels is configured to direct fluid from between the catheter shaft tube and the outer sheath toward the blood pump, and the one or more access channels is configured to direct the one or more elongate components between the drive shaft tube and the catheter shaft tube.

20. The blood pump system of claim 12, wherein the coupling member is fixedly coupled to a driveshaft tube that surrounds the driveshaft.

21.-39. (canceled)
